(19) United States
(12) Patent Application Publication
(10) Pub. No.: US 2014/0348857 A1
(43) Pub. Date: Nov. 27, 2014

(54) METHODS OF DIAGNOSIS AND TREATMENT OF ENDOPLASMIC RETICULUM (ER) STRESS-RELATED CONDITIONS

(71) Applicant: MASSACHUSETTS INSTITUTE OF TECHNOLOGY, Cambridge, MA (US)

(72) Inventors: Paul Chang, Cambridge, MA (US); Miri Jwa, Cambridge, MA (US); Sejal Kamlesh Vyas, Cambridge, MA (US)

(73) Assignee: MASSACHUSETTS INSTITUTE OF TECHNOLOGY, Cambridge, MA (US)

(21) Appl. No.: 14/353,940

(22) PCT Filed: Oct. 26, 2012
(86) PCT No.: PCT/US12/62150

§ 371 Date: Apr. 24, 2014

Related U.S. Application Data
(60) Provisional application No. 61/715,758, filed on Oct. 18, 2012.
(60) Provisional application No. 61/552,210, filed on Oct. 27, 2011.

(51) Int. Cl. G01N 33/573; C07K 16/40; C12N 15/113

(52) U.S. Cl. CPC G01N 33/573; C12N 15/1137; C07K 16/40; G01N 2500/04; G01N 2800/7004
USPC 424/158.1; 514/44 A; 435/7.4; 435/7.21; 435/6.12

(57) ABSTRACT

The present invention relates to methods for treating endoplasmic reticulum (ER) stress-related conditions (e.g., cancer, protein folding/misfolding disease, diabetes mellitus) and for identifying compounds for treating ER stress-related conditions in a subject (e.g., a human). The invention also provides methods for diagnosing an ER stress-related condition in a subject and kits for the treatment of same.

### BACKGROUND OF THE INVENTION

The endoplasmic reticulum (ER) is a multi-functional cellular compartment that functions in protein folding, lipid biosynthesis, and calcium homeostasis. An internal or external cellular insult that compromises ER homeostasis by stressing the protein folding capacity of the ER is termed “ER stress.” Cells cope with ER stress by activating an ER stress signaling network called the Unfolded Protein Response (UPR). The UPR includes at least three components that counteract ER stress: stress gene expression, translational attenuation, and ER-associated protein degradation (ERAD).

Evidence suggests that chronic ER stress is of major importance in the pathogenesis of numerous conditions, such as cancer, protein folding/misfolding disease, myelinating cell-related disease, bipolar disorder, diabetes mellitus, Wolcott-Rallison syndrome, ischemia/reperfusion injury, stroke, neurodegeneration, atherosclerosis, neoplasia, hypoxia, and hypoglycemia. In such diseases, the dysregulation of ER homeostasis leads to cellular dysfunction and, in some instances, cell death.

ER stress-related conditions encompass a number of common, often debilitating or fatal, diseases. For example, the annual incidence of cancer is estimated to be in excess of 1.5 million in the United States alone. Cancer remains the second-leading cause of death in the U.S., accounting for nearly 1 of every 4 deaths. Worldwide, cancer is also a leading cause of death with an annual incidence of over 10 million.

Current therapies available for the treatment of ER stress-related conditions vary considerably depending on the condition being treated. Many of the available therapies are dangerous, costly, toxic, and sometimes ineffective. Thus, there is a need to develop effective alternative therapies for the treatment of ER stress-related conditions, especially ER stress-related conditions that are poorly responsive to current conventional therapies (e.g., metastatic cancers, Alzheimer's disease, amyotrophic lateral sclerosis (ALS)).

### SUMMARY OF THE INVENTION

The present invention is based, at least in part, on the discovery that poly(ADP-ribose) polymerase 16 (PARP-16) functions in the unfolded protein response (UPR) of the endoplasmic reticulum (ER). The invention therefore provides methods for the treatment and diagnosis of ER stress-related conditions.

In a first aspect, the invention features a method of treating a subject with an ER stress-related condition (e.g., cancer), the method including administering to the subject a therapeutically effective amount of a pharmaceutical composition that decreases PARP-16 expression or activity.

In one embodiment of the first aspect, the ER stress-related condition is a cancer, protein folding/misfolding disease, diabetes mellitus, Wolcott-Rallison syndrome, ischemia/reperfusion injury, stroke, neurodegeneration, atherosclerosis, neoplasia, hypoxia, or hypoglycemia. Cancer includes colon adenocarcinoma, esophagus adenocarcinoma, liver hepatocellular carcinoma, squamous cell carcinoma, pancreas adenocarcinoma, islet cell tumor, rectum adenocarcinoma, gastrointestinal stromal tumor, stomach adenocarcinoma, adrenal cortical carcinoma, follicular carcinoma, papillary carcinoma, breast cancer, ductal carcinoma, lobular carcinoma, intraductal carcinoma, mucinous carcinoma, phyllodes tumor, Ewing's sarcoma, ovarian adenocarcinoma, endometrium adenocarcinoma, granulose cell tumor, mucinous cystadenocarcinoma, cervix adenocarcinoma, vulva squamous cell carcinoma, basal cell carcinoma, prostate adenocarcinoma, giant cell tumor of bone, bone osteosarcoma, larynx carcinoma, lung adenocarcinoma, kidney carcinoma, urinary bladder carcinoma, Wilm's tumor, and lymphoma. Protein folding/misfolding diseases include Huntington's disease, spinobulbar muscular atrophy (Kennedy disease), Machado-Joseph disease, dentatorubral-pallidoluysian atrophy (Haw River Syndrome), spinocerebellar ataxia, Alzheimer's disease, Parkinson's disease, amyotrophic lateral sclerosis (ALS), Creutzfeldt-Jakob disease, bovine spongiform encephalopathy (BSE), and light chain amyloidosis (AL).

In a preferred embodiment of the first aspect, the pharmaceutical composition includes a PARP-16-specific inhibitor (e.g., a small molecule, an antibody, or an RNA aptamer).

In a second aspect, the invention features a method of treating a subject with an ER stress-related condition (e.g., multiple sclerosis (MS)), the method including administering to the subject a therapeutically effective amount of a pharmaceutical composition that increases PARP-16 expression or activity.

In one embodiment of the second aspect, the ER stress-related condition is a myelinating cell-related disease, protein folding/misfolding disease, or bipolar disorder. Myelinating cell-related diseases include MS, Charcot-Marie-Tooth disease (CMT), Pelizaeus-Merzbacher Disease (PMD), and Vanishing White Matter Disease (VWMD). Protein folding/misfolding diseases include Huntington's disease, spinobulbar muscular atrophy (Kennedy disease), Machado-Joseph disease, dentatorubral-pallidoluysian atrophy (Haw River Syndrome), spinocerebellar ataxia, Alzheimer's disease, Parkinson's disease, amyotrophic lateral sclerosis (ALS), Creutzfeldt-Jakob disease, bovine spongiform encephalopathy (BSE), and light chain amyloidosis (AL).

In a preferred embodiment of the second aspect, the pharmaceutical composition includes a PARP-16-specific activator.

Typically, the pharmaceutical compositions of the methods of the invention may include a pharmaceutically acceptable carrier and may be administered intramuscularly, intravenously, intradermally, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctival, intravesicularlly, mucosally, intrapericardially, intraumbilically, intraocularally, orally, topically, locally, by inhalation, injection, infusion, continuous infusion, localized perfusion bathing target cells directly, catheter, lavage, in cremes, or lipid compositions.

In a third aspect, the invention features a method of diagnosing an ER stress-related condition (e.g., cancer) in a subject. The method includes analyzing the level of PARP-16 expression or activity in a sample isolated from the subject, where an increased level of PARP-16 expression or activity in the sample relative to the level in a control sample indicates that the subject has the ER stress-related condition. The analyzing may include measuring in the sample the amount of PARP-16 RNA or protein, or mono(ADP-ribosyl)ated PERK, IRE1α, or PARP-16.

In another aspect, the invention provides a method of identifying a candidate compound useful for treating a subject with an ER stress-related condition (e.g., cancer). The method includes contacting a PARP-16 protein, or fragment thereof, with a compound (e.g., a compound selected from a chemical library, an antibody or antibody fragment, an RNA aptamer) and measuring the activity of the PARP-16, where a decrease in PARP-16 activity in the presence of the compound identifies the compound as a candidate compound for treating an ER stress-related condition in a subject.

In a related aspect, the invention provides a method of identifying a candidate compound useful for treating a subject with an ER stress-related condition (e.g., MS). The method includes contacting a PARP-16 protein, or fragment thereof, with a compound (e.g., a compound selected from a chemical library) and measuring the activity of the PARP-16, where an increase in PARP-16 activity in the presence of the compound identifies the compound as a candidate compound for treating an ER stress-related condition in a subject.

In another aspect, the invention provides a kit for treating a subject with an ER stress-related condition. The kit includes a pharmaceutical composition that modulates (e.g., increases or decreases) PARP-16 expression or activity and instructions for administering the pharmaceutical composition to the subject.

Typically, the subject is a mammal, such as a human.

### DEFINITIONS

By “PARP-16-specific activator” is meant an agent that preferentially increases the expression (e.g., mRNA and/or protein level) or at least one biological activity of PARP-16 at least 2-fold (e.g., 3-fold, 5-fold, 10-fold, 20-fold, 30-fold, 50-fold, 100-fold, 200-fold, 300-fold, 500-fold, 1,000-fold, 5,000-fold, or 10,000-fold) greater than a corresponding activity in at least one (e.g., any) other PARP family protein. A PARP-16-specific activator may increase one or more biological activities of a PARP-16 protein including, but not limited to, the ability to attach a mono-ADP-ribose molecule to one or more substrate(s) (e.g., PARP-16, PERK, IRE1α), the ability to localize to the ER membrane, the ability to sense stress in the ER lumen, and the ability to maintain ER structure. More preferably, an observable or measurable increase in activity of PARP-16 is observed upon administration of the activator. A PARP-16-specific activator may alternatively, or additionally, increase the level of PARP-16 nucleic acid or PARP-16 protein. In treatment scenarios, preferably the PARP-16-specific activator is required to produce a therapeutic benefit in the condition being treated (e.g., myelinating cell-related disease, protein folding/misfolding disease, bipolar disorder). A PARP-16-specific activator, for example, includes nucleic acids encoding PARP-16 or the catalytic domain of PARP-16. For example, a PARP activator may be a nucleic acid containing a nucleic acid sequence having at least 80% sequence identity (e.g., at least 85%, 90%, 95%, 96%, 97%, 98%, 99%, or even 100%) to PARP-16.

By “PARP-16-specific inhibitor” is meant an agent that preferentially decreases the expression (e.g., mRNA and/or protein level) or at least one biological activity of PARP-16 at least 2-fold (e.g., 3-fold, 5-fold, 10-fold, 20-fold, 30-fold, 50-fold, 100-fold, 200-fold, 300-fold, 500-fold, 1,000-fold, 5,000-fold, or 10,000-fold) greater than a corresponding activity in at least one (e.g., any) other PARP family protein. A PARP-16-specific inhibitor may decrease one or more biological activities of a PARP-16 protein including the ability to attach a mono-ADP-ribose molecule to one or more substrate(s) (e.g., PARP-16, PERK, IRE1α), the ability to localize to the ER membrane, the ability to sense stress in the ER lumen, and the ability to maintain ER structure. A PARP-16-specific inhibitor may alternatively, or additionally, decrease the level of PARP-16 nucleic acid or PARP-16 protein. In treatment scenarios, preferably the PARP-16-specific inhibitor is required to produce a therapeutic benefit in the condition being treated (e.g., cancer, protein folding/misfolding disease, diabetes mellitus). A PARP-16-specific inhibitor, for example, includes antibodies, or fragments thereof, that specifically bind PARP-16, RNA aptamers (e.g., RNAi molecules), and small molecules.

By the term “RNA aptamer” or “RNAi molecule” is meant a short double-stranded RNA molecule that mediates the down-regulation of a target mRNA (e.g., PARP-16 mRNA) in a cell. An RNAi molecule is typically 15 to 32 (e.g., 17, 18, 19, 20, 21, 22, 23, 24, 25, 26, 27, 28, 29, 30, 31, or 32) nucleotides in length. RNAi molecules include siRNAs, small RNAs, and miRNAs.

By the term “substrate” or “target” is meant a nucleic acid or protein that is bound by one or more (e.g., 1, 2, 3, 4, or 5) PARP protein(s) or PARP fusion protein(s) (e.g., PARP-16 or PARP-16 fusion protein); covalently modified by attachment of a ADP-ribose molecule by the activity of one or more (e.g., 1, 2, 3, 4, or 5) PARP protein(s) or PARP fusion protein(s); or contains a mono- or poly-ADP-ribosyl group that is hydrolyzed by the activity of one or more (e.g., 1, 2, 3, 4, or 5) PARG proteins, PARG fusion proteins, ARH3 proteins, or ARH3 fusion proteins. For example, substrates of PARP-16 include, but are not limited to PERK, IRE1α, and PARP-16. A target protein or substrate protein may localize to different structures or organelles within a cell during different stages of the cell cycle (e.g., interphase, S-phase, prophase, metaphase, telophase, and anaphase) and may, for example, have an activity in the formation of an endoplasmic reticulum stress response.

Throughout this specification and claims, the word “comprise,” or variations such as “comprises” or “comprising,” will be understood to imply the inclusion of a stated integer or group of integers but not the exclusion of any other integer or group of integers.

By “control sample” or “reference sample” is meant any sample, standard, standard curve, or level that is used for comparison purposes. A control sample can be, for example, a prior sample taken from the same subject (e.g., prior to developing symptoms of an ER stress-related condition); a sample from a normal healthy subject (e.g., a subject without an ER stress-related condition); a sample from a subject not having a condition associated with increased levels of PARP-16 expression and/or activity; a sample from a subject that is diagnosed with a propensity to develop a condition associated with increased levels of PARP-16 expression and/or activity, but does not yet show symptoms of the condition; a sample from a subject that has been treated for a condition associated with increased levels of PARP-16 expression and/or activity; or a sample of purified PARP-16 at a known concentration.

The terms “effective amount” or “therapeutically effective amount” refer to a sufficient amount of the agent to provide the desired biological, therapeutic, and/or prophylactic result. That result can be reduction and/or alleviation of the signs, symptoms, or causes of a disease (e.g., cancer, protein folding/misfolding disease) or any other desired alteration of a biological system. For example, a “therapeutically effective amount” when used in reference to treating a cancer refers to an amount of one or more compounds that provides a clinically significant decrease in the cancer, e.g., relieves or diminishes one or more symptoms caused by a condition associated with cancer.

A “pharmaceutically acceptable carrier” is meant a carrier which is physiologically acceptable to a treated mammal (e.g., a human) while retaining the therapeutic properties of the compound with which it is administered. One exemplary pharmaceutically acceptable carrier is physiological saline. Other physiologically acceptable carriers and their formulations are known to one skilled in the art and described, for example, in Remington's Pharmaceutical Sciences (Remington's Pharmaceutical Sciences, 21^{th} ed., A. Gennaro, 2005, Lippincott, Williams & Wilkins, Philadelphia, Pa.), incorporated herein by reference.

A “subject” is a vertebrate, such as a mammal, e.g., a human. Mammals include, but are not limited to, farm animals (such as cows), sport animals, pets (such as cats, dogs, and horses), mice, rats, and primates.

As used herein, and as well understood in the art, “treatment” is an approach for obtaining beneficial or desired results, such as clinical results. Beneficial or desired results can include, but are not limited to, alleviation or amelioration of one or more symptoms or conditions; diminishment of extent of disease, disorder, or condition; stabilization (i.e., not worsening) of a state of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment. The recitation herein of numerical ranges by endpoints is intended to include all numbers subsumed within that range (e.g., a recitation of 1 to 5 includes 1, 1.5, 2, 2.75, 3, 3.80, 4, and 5).

As used herein, “a” or “an” means at least one or one or more unless otherwise indicated. In addition, the singular forms “a,” “an,” and “the,” include plural referents unless the context clearly dictates otherwise. Thus, for example, reference to “a composition containing a therapeutic agent” includes a mixture of two or more therapeutic agents.

Other features and advantages of the invention will be apparent from the following Detailed Description, the drawings, and the claims.

### BRIEF DESCRIPTION OF THE DRAWINGS

The invention may be more completely understood in consideration of the accompanying drawings, which are incorporated in and constitute a part of this specification, and together with the description, serve to illustrate several embodiments of the invention:

FIG. 1A is a set of images showing HeLa cells stained for PARP-16 (green) and organelle markers (red).

FIG. 1B is a depiction showing PARP-16 domain structure. TM=transmembrane domain. PARP=PARP catalytic domain.

FIG. 1C is set of Western immunoblots of a membrane extraction assay showing input, cytosol, supernatant and pellet of 1 M NaCl or 1% Triton X-100 (TX-100) treated membrane fractions. Molecular weight (MW) (kD) at right of blot.

FIG. 1D is a set of images showing HeLa cells co-transfected with mCherry (top row), GFP (second row), GFP-PARP-16 (third row), or mCherry-PARP-16 (bottom row), before and after protease treatments.

FIG. 1E is a set of Western immunoblots of samples from FIG. 1D probed with anti-RFP (left) and anti-GFP (right), showing that the transmembrane domain is C-terminal to the cytoplasmic catalytic domain of PARP-16.

FIG. 1F is a set of images of a protease protection assay using untreated, digitonin-treated, or Proteinase K-treated cells expressing GFP-PARP-16 or GFP.

FIG. 1G is a set of diagrams of PARP-16 mutant proteins (left) and images of HeLa cells transfected with GFP fusions of each mutant, before and after Digitonin treatment (right).

FIG. 1H is a schematic diagram showing PARP-16 topology relative to the ER membrane.

FIG. 2A is a set of images of HeLa cells untransfected or transfected with siRNA against PARP-16 and stained for PARP-16 or Calnexin.

FIG. 2B is a set of images showing PARP-16 knockdown HeLa cells formaldehyde-fixed and stained using DiOC₁₈ for ER visualization.

FIG. 2C is an autoradiogram of pure recombinant GST-PARP-16 and GST-PARP-16^{H152Q Y182A} following NAD⁺ incorporation assays, showing PARP-16 synthesizes mono(ADP-ribose) (mADPr) and undergoes automodification. Asterisk=MW of GST-PARP-16.

FIG. 2D is an autoradiogram and Western immunoblot of an ER microsome (ADP-ribosyl)ation Assay (EMAA) using GFP-PARP-16 or GFP-PARP-16^{H152Q Y182A} containing microsomes. Asterisk=high MW incorporation of ³²P-NAD.

FIG. 2E is a set of images of HeLa cells expressing GFP-PARP-16, GFP-PARP-16^{H152Q Y182A}, or GFP-PARP-16^{Cb5} for 16 h or 28 h, or untransfected cells treated with Brefeldin A (BFA), stained for PARP-16 (green) and Calnexin (red).

FIG. 2F is a set of graphs showing the effects of Tunicamycin, Thapsigargin, or Brefeldin A treatment on PARP-16 knock-down cells (top) and a representative Western immunoblot of PARP-16 expression upon siRNA knock-down (bottom). Trypan blue staining was performed for control or PARP-16 knock-downs at indicated time points after Tunicamycin, Thapsigargin or Brefeldin A treatment. 16.3 and 16.4 are different siRNAs against PARP-16 (n=4 for siRNA 16.3, and 2 for siRNA 16.4). For Tunicamycin, Thapsigargin, or Brefeldin A-treated PARP-16 knock-down cells, 0.001<p<0.05.

FIG. 3A is an image of purified ER microsomes stained with ER-Tracker Red.

FIG. 3B is a set of Western immunoblots of purified ER microsomes immunoblotted for indicated proteins. RER=Rough ER.

FIG. 3C is a Coomassie-stained gel showing amounts of GST-PARP-16 and GST-PARP-16^{H152Q Y182A} utilized for reaction shown in FIG. 2A. Asterisk=full-length proteins.

FIG. 4A is a set of images of cells expressing GFP-PARP-16, GFP-PARP-16^{H152Q Y182A}, or GFP-PARP-16^{Cb5} for 16 h or 28 h stained for GFP-PARP-16 (green), Calnexin (red; left), PERK (red; right), and DNA (blue), showing that prolonged overexpression of PARP-16 causes abnormal ER structures.

FIG. 4B is a set of Western immunoblots showing the expression levels of GFP fusion proteins in each condition of FIG. 4A. n=3.

FIG. 4C is a graph quantifying the percentage of abnormal ER structures in each condition of FIG. 4A.

FIG. 5A is a graph showing the effects of PARP-16 knock-down on reactive oxygen species (ROS) in control and PARP-16 knock-downs. Shown are values in arbitrary unit (A.U.) for fluorescence intensity of CM-H₂DCFDA before and after H₂O₂ treatment. n=2; p>0.05 after H₂O₂ treatment.

FIG. 5B is a graph showing the intracellular Ca²⁺ concentration in control and PARP-16 knock-downs. Shown are values in arbitrary unit (A.U.) for fluorescence intensity ratio 340 nm/380 under basal conditions (time points 1-3), during Thapsigargin treatment (time points 4-7), and after EGTA addition (time points 8-11). Each time points are 2 min apart. n=2; p>0.1 after Thapsigargin treatment.

FIG. 5C is a set of images showing cells treated with or without cisplatin and immuno-stained for γ-H2AX (red) and DNA (blue) (top), and γ-H2AX positive cells were counted (bottom). n=2; p>0.1 in PARP-16 knock downs.

FIG. 5D is a set of images showing cells treated with or without arsenite and immuno-stained for TIA-1 (red) and DNA (blue) (top), and TIA-1 positive cells were counted (bottom). n=2; p<0.05 in PARP-16 knock downs.

FIG. 6A is a set of autoradiogram and Western immunoblots showing extracted and immunoprecipitated ER microsomes from HeLa cells overexpressing GFP-PARP-16 and treated without (UT), or with Brefeldin A (BFA), thapsigargin (TG), or tunicamycin (TUN) subjected to NAD⁺ incorporation assays. Asterisk=high MW NAD⁺ incorporation. n=5; 0.01<p of fold increase <0.05 for all stressors.

FIG. 6B is a set of Western immunoblots showing ER microsome based co-immunoprecipitation assays of GFP-fusion proteins. Shown are immunoblots of precipitated GFP fusions.

FIG. 6C is a set of autoradiogram and Western immunoblots of EMAAs showing GFP-PERK immunoprecipitates. n=4; 0.005<p of fold increase <0.05 for all stressors. PARP-16 immunoblots of control and PARP-16 knock-down lysates are shown.

FIG. 6D is a set of autoradiogram and Western immunoblots of EMAAs showing GFP-IRE 1α immunoprecipitates. n=4; 0.005<p of fold increase <0.05 for all stressors. PARP-16 immunoblots of control and PARP-16 knock-down lysates are shown.

FIG. 6E is a set of images showing that recombinant PARP-16 can mono-ADP-ribosylate PERK in standard NAD⁺ incorporation assays using ER microsome-purified GFP-PERK and bacterially purified GST-PARP-16 and PARP-16^{H152Q Y182A}.

FIG. 6F is a set of autoradiogram and Western immunoblots of EMAAs for SEC61β, ATF6, and PARP-16, showing the immunoprecipitated GFP fusion proteins. n=2.

FIG. 7A is a set of Western immunoblots cells overexpressing GFP or GFP fusions to PARP-16 or PARP-16^{H152Q/Y182A} (left) or cells transfected with control or PARP-16 siRNA (right). MW (kD) at right of blots. UT=untreated; BFA=Brefeldin A; TG=Thapsagargin; TUN=Tunicamycin; PERK^{p}=phospho-PERK; eIF2α^{p}=phospho-eIF2α.

FIG. 7B is an agarose gel showing undigested and PstI-digested XBP-1 cDNA amplified by RT-PCR from mRNA of HeLa cells overexpressing GFP-PARP-16, GFP-PARP-16^{H152Q Y182A}, or total mRNA of control or PARP-16 knockdown cells, treated or untreated with tunicamycin. Unspliced (U) and spliced (S) XBP-1 cDNA were amplified via RT-PCR then cut with Pst1 restriction enzyme. Only unspliced XBP-1 is cut by PstI. Asterisk=hybrid amplicons.

FIG. 7C is a set of Western immunoblots of cell lysates treated with control or PARP-16 siRNA and Tunicamycin (left) and RT-qPCR analysis of UPR-dependent transcription in control or PARP-16 knock-downs treated with Tunicamycin (right).

FIG. 7D is an autoradiogram of ER microsome based (ADP-ribosyl)ation (right) and kinase assays (left) wherein microsomes containing GFP-PERK were (ADP-ribosyl)ated via addition of 0.5 μg GST-PARP-16 or GST-PARP-16^{H152Q Y182A} in the presence of ³²P-NAD⁺. Duplicate NAD⁺ incorporation reactions were performed under identical conditions using unlabeled NAD⁺, then kinase activity assayed via ³²P-ATP incorporation. n=4.

FIG. 7E is an autoradiogram of ER microsome based (ADP-ribosyl)ation (right) and kinase assays (left) wherein microsomes containing GFP-IRE 1α were (ADP-ribosyl)ated via addition of 0.5 μg GST-PARP-16 or GST-PARP-16^{H152Q Y182A} in the presence of ³²P-NAD⁺. Duplicate NAD⁺ incorporation reactions were performed under identical conditions using unlabeled NAD⁺, then kinase activity assayed via ³²P-ATP incorporation. n=4.

FIG. 7F is an autoradiogram of ER microsome based (ADP-ribosyl)ation and IRE1α endonuclease assays using unlabeled NAD⁺ for (ADP-ribosylation of GFP-IRE1α. In vitro transcribed ³²P labeled XBP-1 transcript was incubated with the (ADP-ribosyl)ated GFP-IRE1α immunoprecipitates and assayed for splicing as indicated via presence of 5′- and 3′-exons. n=4.

FIG. 8A is a full image of the agarose gel shown in FIG. 7B. Asterisks=290 and 183 bp fragments originated from unspliced XBP-1 cDNA, upon digestion with PstI restriction enzyme. Triangle represents hybrid amplicons. (OE)=over-expression; (ctrl)=control; (P-16)=PARP-16; (U)=unspliced; (S)=spliced; (UT)=untreated; and (TUN)=Tunicamycin treated.

FIG. 8B is a full image of the agarose gel shown in FIG. 10C. Asterisks=290 and 183 bp fragments originated from unspliced XBP-1 cDNA, upon digestion with PstI restriction enzyme. Triangle represents hybrid amplicons. (OE)=over-expression; (ctrl)=control; (P-16)=PARP-16; (U)=unspliced; (S)=spliced; (UT)=untreated; (BFA)=Brefeldin A treated; and (TUN)=Tunicamycin treated.

FIG. 8C is a set of autoradiograms of ER microsome based NAD⁺ incorporation and kinase assays. ER microsomes containing GFP-PERK were (ADP-ribosyl)ated using either GST-PARP-16 or GST-PARP-16^{H152Q Y182A} in the presence of ³²P-NAD⁺. The duplicate NAD⁺ incorporation reactions were performed under the same conditions using unlabeled NAD⁺ instead, and then subjected to kinase assays using ³²P-ATP.

FIG. 9A is a set of Western immunoblots of showing that CD3δ-YFP, a model substrate of ERAD machinery, exhibited similar degradation kinetics in PARP-16 knock-downs and controls as assayed by cycloheximide chase.

FIG. 9B is a set of Western immunoblots of a similar ERAD activity assay as in FIG. 9A, except that the cells used co-expressed CD3δ-YFP and a mCherry fusion to either PARP-16 or PARP-16^{H152Q Y182A}.

FIG. 9C is a set of Western immunoblots showing that the protein-folding capacity of the ER in PARP-16 knock-downs (P-16) appear to be similar to controls (Ctrl) as the protein concentrations of ER chaperones BiP and Calnexin (CNX), and disulfide isomerases PDI and ERp57, were similar. Tubulin is a loading control.

FIG. 10A is a set of Western immunoblots of ER microsome based co-immunoprecipitation assays in which GFP-PERK or GFP-IRE1α are purified from ER microsomes from cells treated with control (Ctrl) or PARP-16 (P-16) siRNA to assess BiP binding. PARP-16 blots from each assay are shown.

FIG. 10B is a set of Western immunoblots probed for the indicated protein or phosphorylated variant from cells expressing GFP, or GFP fusions to PARP-16 or PARP-16^{Cb5}, untreated (UT), or treated with Brefeldin A (BFA) or Tunicamycin (TUN).

FIG. 10C is an agarose gel showing a XBP-1 mRNA splicing assay from control, GFP-PARP-16, or GFP-PARP-16^{H152Q Y182A} expressing cells, or cells transfected with control or PARP-16 siRNA, untreated (UT) or treated with Tunicamycin (TUN). Unspliced (U) and spliced (S) XBP-1 cDNA were amplified via RT-PCR then cut with Pst1 restriction enzyme. Only unspliced XBP-1 is cut by PstI, Asterisk=hybrid amplicons.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF THE INVENTION

We have discovered that poly(ADP-ribose) polymerase 16 (PARP-16) functions in the unfolded protein response (UPR) of the endoplasmic reticulum (ER). Specifically, we found that PARP-16 activates two main sensors of ER stress, PERK and IRE1α, during the UPR. PARP-16 therefore initiates and transduces ER stress signals from the ER lumen to the cytoplasm. Accordingly, the invention provides methods and kits for the treatment of ER stress-related conditions by modulation of PARP-16 expression or function. The invention also provides methods for diagnosing an ER stress-related condition in a subject. Lastly, the invention provides screening methods for the identification of candidate compounds that may be useful for treating an ER stress-related condition.

### Poly(ADP-Ribose) Polymerase (PARP)

PARP is also known as poly(ADP-ribose) synthase and poly-ADP-ribosyltransferase. PARP catalyzes the formation of mono(ADP-ribose) (mADPr) and poly(ADP-ribose) (pADPr) modifications of cellular proteins (as well as itself), and thereby modifies the activities of its substrate proteins. mADPr or pADPr is attached by PARP onto substrate proteins in a process that requires nicotinamide adenine dinucleotide (NAD⁺). Seventeen members of the PARP family of genes are present in the mammalian genome.

PARPs play a role in the general physiology of the cell, including the regulation of transcription, cell proliferation, and chromatin remodeling (see D′ amours et al., Biochem. 342: 249-268, 1999). PARP knockouts in Drosophila melanogaster are embryonic lethal (Tulin et al., Genes Dev. 16: 2108-2119, 2002). PARPs also function during conditions of cellular stress such as DNA damage repair. More recently, we identified that certain PARPs play a role in cytoplasmic stress granule assembly and disassembly. Here, we identify that PARP-16, in particular, is required for the UPR of the ER. In being required to activate two key sensors of ER stress, PERK and IRE1α, PARP-16 plays a critical role in sensing ER stress and regulating cellular responses to ER stress.

### Measuring PARP-16 Biological Activity

We have found that decreasing PARP-16 expression or activity results in decreased activation of both the PERK and IRE1α arms of the UPR during ER stress. On this basis, modulating (e.g., decreasing) PARP-16 expression or activity can be used for treating ER stress-related conditions (e.g., cancer). In addition, PARP-16 expression or activity levels may be indicative of an ER stress-related condition (e.g., cancer) in a subject.

The biological activity of PARP-16 includes, but is not limited to, the ability to covalently attach an ADP-ribose molecule to a substrate (e.g., a protein, a RNA molecule, a DNA molecule, or a lipid), the ability to localize to the cell nucleus, the ability to localize to the ER (e.g., the ER membrane), the ability to function in the PERK arm of the UPR (e.g., by mono(ADP-ribosyl)ation of PERK), the ability to function in the IRE1α arm of the UPR (e.g., by mono(ADP-ribosyl)ation of IRE1α), the ability to auto-modify itself with mADPr, the ability to sense stress in the ER lumen, and the ability to maintain ER structure. Other PARP proteins respond differently to cellular stress. For example, PARP5A, PARP12, PARP13.1, PARP13.2, and PARP 15 have the ability to localize to stress granules and the ability to promote stress granule formation; PARP11 has the ability to localize to stress granules and the ability to promote disassembly of stress granules; and PARP 13.1 has the ability to decrease the activity of RNAi and the ability to add one or more ADP-ribose molecules to Argonaut. In the diagnostic methods described herein, these activities can be measured using any appropriate assay known in the art, such as those described below.

Assays to measure the ability of PARP-16 to covalently attach an ADP-ribose to one or more substrate(s) (e.g., a protein, a RNA, a DNA, or a lipid) involve the incubation of PARP-16 with the one or more substrate(s) in the presence of a labeled NAD⁺ molecule (e.g., radiolabeled, fluorescently-labeled, and colorimetrically-labeled NAD⁺). A radiolabeled NAD⁺ substrate may contain one or more radioisotopes including, but not limited to, C¹⁴ (e.g., C¹⁴-adenine), P³², and H³. Additional NAD⁺ substrates include fluorescently-labeled NAD⁺ (Putt et al., Anal. Biochem. 78: 326, 2004), colorimetrically-labeled NAD⁺ (Nottbohn et al., Agnew. Chem. Int. Ed. 46: 2066-2069, 2007), and biotinylated NAD⁺ (6-biotin-17-NAD; R & D Systems). Following incubation of PARP-16 with the labeled NAD⁺ and one or more substrate molecules (e.g., PERK, IRE1α), the specific labeling of the substrate with mADPr is determined by measuring the amount of the label associated with the NAD⁺ covalently bound to the one or more substrates. An increase in the amount of the label associated with the NAD⁺ covalently bound to the one or more substrate(s) indicates PARP-16 activity.

In another example of a PARP assay, the automodification of PARP-16 is measured by incubating PARP-16 with a labeled NAD⁺ substrate and subsequently, measuring the amount of the label associated with the NAD⁺ covalently bound to PARP-16. An increase in the amount of the label associated with the NAY covalently bound to PARP-16 indicates PARP-16 automodification.

In an alternative assay, PARP-16 may be incubated with one or more substrates (e.g., PERK and/or IRE1α) and non-labeled NAD⁺. The mono-ADP-ribosylation of the one or more substrates may be measured by contacting the one or more substrates with an ADP-ribose antibody. For example, a sample of substrate proteins may be electrophoresed and immmunoblotted with an anti-ADP-ribose antibody. An increased number of proteins or an increased level of detection using an anti-ADP-ribose antibody indicates an increase in the activity of PARP-16.

Assays to measure the ability of a PARP to localize to a specific cellular structure or organelle using immunofluorescence microscopy are known in the art. For example, antibodies specific for a PARP or PARP fusion protein and antibodies specific for one or more proteins specific for a cellular structure or organelle (e.g., cytoskeleton, mitochondria, trans-Golgi network, endoplasmic reticulum, early endosome, centrosome, GW bodies, nuclear envelope, lysosome, peroxisomes, histones, Cajal bodies, nucleus, and mitochondria) may be used to perform immunofluorescent microscopy. Localization of a PARP or PARP fusion protein may be measured in high-throughput experiments by co-localization of PARP or PARP fusion proteins (e.g., PARP-16) with one or more proteins specific for a cellular structure or organelle (e.g., Calnexin, an ER marker). Localization of PARP in the nucleus may also be demonstrated by co-localization of a dye that stains DNA (e.g., 4′,6-diamindino-2-phenylindole (DAPI)) and an antibody that specifically binds a PARP or PARP fusion proteins.

Localization of a PARP to a specific cell structure or organelle may occur only during one or more (e.g., 1, 2, 3, 4, 5, 6, 7, 8, or 9) specific stages of the cell cycle, including, but not limited to, G1, S, G2, prophase, prometaphase, metaphase, anaphase, telophase, and cytokinesis stages. For the purposes described herein, a PARP is deemed to have the ability to localize to a specific cellular structure or organelle if it localizes to the specific cellular structure or organelle in at least one stage (e.g., prophase) of the cell cycle.

The ability of PARP-16 to function in the UPR can be measured using Western immunoblotting and/or autoradiography. For example, ER microsomes are purified from cells expressing PARP-16 (e.g., recombinant GFP-tagged PARP-16) and then tested in a standard NAD⁺ incorporation assay utilizing ³²P-NAD⁺. PARP-16 and/or other PARP-16 substrates (e.g., PERK, IRE1α) can then be analyzed for mADPr modification by Western immunoblot (e.g., by a change in migration of protein) and/or by autoradiography (e.g., by ³²P-NAD⁺ incorporation). In such assays, increased mADPr modification by PARP-16 may be induced by exposure to ER stress conditions, for example, by treatment with brefeldin A (BFA), thapsigargin (Tg), and/or tunicamycin (Tun).

Any of the above-referenced PARP activity assays may be performed to determine the activity of a PARP protein (e.g., PARP-16). In addition, the biological activity of a PARP (e.g., PARP-16) may be assessed using any of the above-described cellular or in vitro assays.

### PARP Inhibitors

Any PARP inhibitor, such as a PARP-16-specific inhibitor, may be used in the methods described herein. Exemplary inhibitors include RNA aptamers (RNAi molecules), PARP-16-specific antibodies, and small molecule inhibitors.

### RNA Aptamers

Any appropriate RNA aptamer may be used in the present invention. The design and therapeutic effectiveness of RNA aptamers (e.g., siRNA, small RNA, shRNA) is described in McCaffrey et al. (Nature 418:38-39, 2002). RNA aptamers are at least 15 nucleotides, preferably, 17, 18, 19, 20, 21, 22, 23, 24, 25, 26, 27, 28, 29, 30, 31, 32, 33, 34, or 35 nucleotides in length and even up to 50 or 100 nucleotides in length (inclusive of all integers in between). An RNA aptamer may target any part of the sequence encoding the target protein (e.g., any part of an mRNA encoding PARP-16). Non-limiting examples of RNA aptamers are at least 80% identical (e.g., at least 85%, 90%, 95%, 96%, 97%, 98%, 99%, or even 100% identical) to or complementary to the translational start sequence or the nucleic acid sequence encoding the first 10, 20, 30, 40, 50, 60, 70, 80, 90, or 100 amino acids of a target protein (e.g., PARP-16).

The specific requirements and modifications of small RNA are known in the art and are described, for example in PCT Publication No. WO01/75164, and U.S. Application Publication Nos. 2006/0134787, 2005/0153918, 2005/0058982, 2005/0037988, and 2004/0203145, the relevant portions of which are herein incorporated by reference. siRNAs can also be synthesized or generated by processing longer double-stranded RNAs, for example, in the presence of the enzyme dicer under conditions in which the dsRNA is processed to RNA molecules of about 17 to about 26 nucleotides. siRNAs can also be generated by expression of the corresponding DNA fragment (e.g., a hairpin DNA construct). Generally, the siRNA has a characteristic 2- to 3-nucleotide 3′ overhanging ends, preferably these are (2′-deoxy) thymidine or uracil. The siRNAs typically comprise a 3′ hydroxyl group. Single-stranded siRNAs or blunt-ended dsRNA may also be used. In order to further enhance the stability of the RNA, the 3′ overhangs may be stabilized against degradation. For example, the RNA may be stabilized by including purine nucleotides, such as adenosine or guanosine. Alternatively, substitution of pyrimidine nucleotides by modified analogs, e.g., substitution of uridine 2-nucleotide overhangs by (2′-deoxy)thymidine is tolerated and does not affect the efficiency of RNAi. The absence of a 2′ hydroxyl group significantly enhances the nuclease resistance of the overhang in tissue culture medium.

siRNA molecules can also be obtained through a variety of protocols including chemical synthesis or recombinant production using a Drosophila in vitro system. They can be commercially obtained from companies such as Dharmacon Research Inc. or Xeragon Inc., or they can be synthesized using commercially available kits such as the Silencer™ siRNA Construction Kit from Ambion (catalog number 1620) or HiScribe™ RNAi Transcription Kit from New England BioLabs (catalog number E2000S).

Alternatively siRNA can be prepared using standard procedures for in vitro transcription of RNA and dsRNA annealing procedures such as those described in Elbashir et al. (Genes & Dev., 15:188-200, 2001), Girard et al. (Nature 442:199-202, 2006), Aravin et al. (Nature 442:203-207, 2006), Grivna et al. (Genes Dev. 20:1709-1714, 2006), and Lau et al. (Science 313:305-306, 2006). siRNAs may also be obtained by incubation of dsRNA that corresponds to a sequence of the target gene in a cell-free Drosophila lysate from syncytial blastoderm Drosophila embryos under conditions in which the dsRNA is processed to generate siRNAs of about 21 to about 23 nucleotides, which are then isolated using techniques known to those of skill in the art. For example, gel electrophoresis can be used to separate the 21-23 nt RNAs and the RNAs can then be eluted from the gel slices. In addition, chromatography (e.g., size exclusion chromatography), glycerol gradient centrifugation, and affinity purification with antibody can be used to isolate the small RNAs.

Short hairpin RNAs (shRNAs), as described in Yu et al. (Proc. Natl. Acad. Sci. U.S.A. 99:6047-6052, 2002) or Paddison et al. (Genes & Dev. 16:948-958, 2002), incorporated herein by reference, may also be used. shRNAs are designed such that both the sense and antisense strands are included within a single RNA molecule and connected by a loop of nucleotides (3 or more). shRNAs can be synthesized and purified using standard in vitro T7 transcription synthesis as described above and in Yu et al. (supra). shRNAs can also be subcloned into an expression vector that has the mouse U6 promoter sequences which can then be transfected into cells and used for in vivo expression of the shRNA.

PARP-16 RNA aptamers are available commercially and include, for example, PARP-16 miRNA (OriGene Technologies, USA), PARP-16 shRNA (OriGene Technologies, USA), and PARP-16 siRNA duplexes (OriGene Technologies, USA).

A variety of methods and reagents are available for transfection, or introduction, of dsRNA into mammalian cells including but not limited to: TransIT-TKO™ (Minis, Cat. # MIR 2150), Transmessenger™ (Qiagen, Cat. #301525), Oligofectamine™ and Lipofectamine™ (Invitrogen, Cat. # MIR 12252-011 and Cat. #13778-075), siPORT™ (Ambion, Cat. #1631), and DharmaFECT™ (Fisher Scientific, Cat. # T-2001-01). Agents are also commercially available for electroporation-based methods for transfection of siRNA, such as siPORTer™ (Ambion Inc, Cat. ##1629). Microinjection techniques can also be used. The small RNA can also be transcribed from an expression construct introduced into the cells, where the expression construct includes a coding sequence for transcribing the small RNA operably-linked to one or more transcriptional regulatory sequences. Where desired, plasmids, vectors, or viral vectors can also be used for the delivery of dsRNA or siRNA and such vectors are known in the art. Protocols for each transfection reagent are available from the manufacturer. Additional methods are known in the art and are described, for example in U.S. Patent Application Publication No. 20060058255.

Assays for measuring RNA aptamer activity in a cell are also known in the art. For example, psiCHECK™-1 and psiCHECK™-2 assays systems provide methods for the measurement of RNA aptamer activity in a cell. In these assays systems, Renilla luciferase is used a primary reporter gene and a target gene (e.g., PARP-16) is cloned a multiple cloning region located downstream of the Renilla translational stop codon. Initiation of the RNAi process towards the target gene (e.g., PARP-16) results in the cleavage and subsequent degradation of the fusion mRNA encoded by the psiCHECK vectors. Measurement of decreased Renilla luciferase activity in the cells indicates a decrease in the activity of RNAi in the cell. In experiments using the psiCHECK assay system, a cell is treated with an inhibitor of one or more PARP (e.g., PARP-16) and one or more RNAi molecules.

### PARP-Specific Antibodies

Antibodies specific to the one or more (e.g., 1, 2, 3, 4, 5, 6, 7, 8, 9, 10, 11, 12, 13, 14, 15, 16, or 17 or more) PARP or PARP fusion proteins (e.g., PARP-16 or a PARP-16 fusion protein, e.g., GFP-tagged PARP-16) can be generated using standard methods, such as those described herein. Antibodies specific for one or more PARP or PARP fusion proteins may be used in quantitative assays to measure to amount of one or more (e.g., 1, 2, 3, 4, 5, 6, 7, 8, 9, 10, 11, 12, 13, 14, 15, 16, or 17 or more) PARP proteins present in a cell, cell lysate, biological sample, or extracellular medium. Antibodies specific to the one or more (e.g., 1, 2, 3, 4, 5, 6, 7, 8, 9, 10, 11, 12, 13, 14, 15, 16, or 17 or more) PARP or PARP fusion proteins may also be used to identify specific binding partners or potential inhibitors or activators of the one or more PARP and/or PARP fusion proteins.

PARP-16 antibodies, for example, are available commercially and include GWB-30FD2D (GenWay Biotech, Inc., USA), ab84641 (Abeam, USA), ab104023 (Abeam, USA), ab117855 (Abeam, USA), and O-25 (Santa Cruz Biotechnology, Inc., USA).

Furthermore, antibodies can be prepared using any method known in the art. For the preparation of polyclonal antibodies reactive with one or more PARP (e.g., PARP-16), PARP fusion proteins (e.g., GST-PARP-16), fragments of PARP (e.g., PARP-16 deletion mutant, e.g., PARP-16^{ΔC}, see below), and/or fragments of PARP fusion protein(s) can be purified from natural sources (e.g., cultures of cells expressing one or more PARP proteins, e.g., PARP-16) or synthesized in, e.g., mammalian, insect, or bacterial cells by expression of corresponding DNA sequences contained in a suitable cloning vehicle (e.g., the nucleic acids encoding PARP-16 and PARP-16 fusion proteins) using techniques that are standard in the art.

PARP-16-specific antibodies preferably bind to the cytoplasmic domain (e.g., the catalytic domain) of PARP-16. For example, the PARP-16-specific antibody may bind at or near the PARP-16 active site or at an epitope that, once bound by the antibody, renders the PARP-16 protein inactive. PARP-16-specific antibodies, may have a Kd for PARP-16 of at least about 10 μM, alternatively at least about 1 μM, alternatively at least about 100 nM, alternatively at least about 10 nM, alternatively at least about 1 nM, or greater.

Alternatively, monoclonal antibodies can be produced using hybridoma technology, which involves removing the spleen from the inoculated animal, homogenizing the spleen tissue, and suspending the spleen cells suspended in phosphate buffered saline (PBS), which are then fused with permanently growing myeloma partner cells, and the products of the fusion plated into a number of tissue culture wells in the presence of selective agents, such as hypoxanthine, aminopterine, and thymidine (Mocikat, J. Immunol. Methods. 225: 185-189, 1999; Srikumaran et al., Science. 220: 522, 1983). The wells can then be screened using standard techniques to identify those containing cells making antibody capable of binding to the desired PARP protein and the antibody can subsequently be purified from those cells using well-known techniques.

As an alternate or adjunct immunogen to a PARP protein and/or PARP fusion protein, peptides corresponding to relatively unusual regions of a PARP protein or PARP fusion protein can be generated and coupled to keyhole limpet hemocyanin (KLH) through an introduced C-terminal lysine. Antiserum to each of these peptides can be similarly affinity-purified on peptides conjugated to BSA, and specificity tested by ELISA and Western blotting using peptide conjugates, and by Western blotting and immunoprecipitation using a PARP protein, PARP fusion protein, and/or fragment of a PARP protein or fusion protein.

Antibodies of the invention are desirably produced using PARP protein and/or PARP fusion protein amino acid sequences that do not reside within highly conserved regions, and that appear likely to be antigenic, as evaluated by criteria such as those provided by the Peptide Structure Program (Genetics Computer Group Sequence Analysis Package, Program Manual for the GCG Package, Version 7, 1991) using the algorithm of Jameson et al., CABIOS 4:181, 1988. These fragments can be generated by standard techniques, e.g., by PCR, cloned into any appropriate expression vector, and used to generate antibodies as described above or as known in the art.

In addition to intact monoclonal and polyclonal anti-PARP or anti-PARP fusion protein antibodies, various genetically engineered antibodies and antibody fragments (e.g., F(ab′)2, Fab′, Fab, Fv, and sFv fragments) can be produced using standard methods.

### Small Molecule Inhibitors

Small molecules that inhibit one or more PARP family protein(s) are known in the art. Examples of small molecule inhibitors of PARP include, but are not limited to, 3-aminobenzamide, 4-[[3-[4-(cyclopropanecarbonyl)piperazine-1-carbonyl]-4-fluorophenyl]methyl]-2H-phthalazin-1-one (Olaparib), 4-iodo-3-nitrobenzamide (Iniparib), 2-[(2R)-2-methylpyrrolidin-2-yl]-1H-benzimidazole-4-carboxamide (ABT-888), 8-Fluoro-2-{4-[(methylamino)methyl]phenyl}-1,3,4,5-tetrahydro-6H-azepino[5,4,3-cd]indol-6-one (AG014699), and 4-methoxy-carbazole (CEP 9722), 2-[4-[(3S)-piperidin-3-yl]phenyl]indazole-7-carboxamide hydrochloride (MK 4827). Small molecule inhibitors of PARP also include derivatives or analogs of known PARP inhibitors. Derivatives of 3-aminobenzamide, Olaparib, Iniparib, and CEP 9722 are described, for example, in PCT Publication Nos. WO2009/064738, WO2004/080976, WO1996/022791, and WO2008/063644. Other small molecule inhibitors of PARP are described, for example, in PCT Publication No. WO2008/030887.

Additionally, small molecule inhibitors specific for a particular PARP (e.g., PARP-16) can rationally designed. Design as disclosed herein can include knowing or predicting the three-dimensional shape (“conformation”) of the binding domain or active site of the PARP protein, and also controlling and/or predicting the conformation of the drug, i.e., a candidate PARP inhibitor (e.g., PARP-16-specific small molecule inhibitor) that is to interact with the binding domain of the PARP protein (e.g., PARP-16).

Determining the conformation of the active site of a target PARP protein (e.g., PARP-16) can help in identifying binding of the PARP inhibitors in the active site of the target PARP protein. For example, to design a PARP-16-specific small molecule inhibitor, a related protein of known structure (e.g., PARP 12, another predicted mono-ADP(ribosyl)transferase; PDB 1D code: 2PA9) can be used to model a predicted three-dimensional structure of the PARP-16 active site. Preferably, a known PARP inhibitor (e.g., a small molecule inhibitor described above) can be used to evaluate binding within the predicted active site of the target PARP protein (e.g., PARP-16).

Based on this evaluation, computational techniques for drug design are used to design candidate small molecule inhibitors for the specific target PARP (e.g., PARP-16) based on the structure of a known PARP inhibitor and the predicted structure of active site of the target PARP. The known PARP inhibitor molecule can be examined through the use of computer modeling using a docking program such as GRID, DOCK, or AUTODOCK (see, e.g., Fischer, Anal Bioanal Chem. 375: 23-25, 2003). This procedure can include computer fitting of a three-dimensional structure of the PARP inhibitor molecule to a binding domain of the PARP protein to ascertain how well the shape and the chemical structure of the known PARP inhibitor molecule will complement the active site of the target PARP protein. Computer programs can also be employed to estimate the attraction, repulsion, and steric hindrance of the known PARP inhibitor to the binding domain of the PARP protein. Typically, the tighter the fit (e.g., the lower the steric hindrance, and/or the greater the attractive force) the more potent the PARP inhibitor will be since these properties are consistent with a tighter binding constant. Preferably, candidate small molecule inhibitors for the specific target PARP (e.g., PARP-16) are rationally designed using the structures of the known PARP inhibitors as scaffolds. The more specificity in the design of a candidate PARP inhibitor for the known or predicted active site of the target PARP, the more likely it can be that the candidate PARP inhibitor will not interfere with other properties of the target PARP protein or other proteins. This can minimize potential side-effects due to unwanted interactions with other proteins.

Numerous computer programs are available and suitable for rational drug design and the processes of computer modeling, model building, and computationally identifying, selecting, and evaluating candidate PARP inhibitors using the methods described herein. These include, for example, GRID (available from Oxford University, UK), MCSS (available from Molecular Simulations Inc., Burlington, Mass. USA), AUTODOCK (available from Oxford Molecular Group, UK), FLEX X (available from Tripos, St. Louis, Mo. USA), DOCK (available from University of California, San Francisco, USA), CAVEAT (available from University of California, Berkeley, USA), HOOK (available from Molecular Simulations Inc., Burlington, Mass. USA), and 3D database systems such as MACCS-3D (available from MDL Information Systems, San Leandro, Calif. USA), UNITY (available from Tripos, St. Louis, MO USA), ICM (available from Molsoft LLC, La Jolla, Calif. USA), and CATALYST (available from Molecular Simulations Inc., Burlington, Mass. USA).

Candidate PARP-specific small molecule inhibitors can also be computationally designed “de novo” using such software packages as LUDI (available from Biosym Technologies, San Diego, Calif. USA), LEGEND (available from Molecular Simulations Inc., Burlington, Mass. USA), and LEAPFROG (Tripos Associates, St. Louis, Mo. USA). Compound deformation energy and electrostatic repulsion, can be evaluated using programs such as GAUSSIAN 92, AMBER, QUANTA/CHARMM, AND INSIGHT II/DISCOVER. These computer evaluation and modeling techniques can be performed on any suitable hardware including, for example, workstations available from Silicon Graphics, Sun Microsystems, and the like.

Alternatively, candidate small molecule inhibitors specific for a particular PARP can be synthesized and formed into a complex with the target PARP (e.g., PARP-16), and the complex can then be analyzed by x-ray crystallography to identify the actual position of the bound candidate PARP inhibitor. The structure and/or functional groups of the candidate PARP inhibitor can then be adjusted, if necessary, in view of the results of the x-ray analysis, and the synthesis and analysis sequence repeated until an optimized PARP inhibitor is obtained.

### Methods for Identification of Candidate Compounds Useful for Treating an ER Stress-Related Condition

The methods of the invention may be used to identify one or more candidate compounds useful for treating a subject with an ER stress-related condition. These candidate compounds include PARP-16-specific activators and inhibitors. PARP-16, or a fragment thereof, is contacted with a compound (e.g., a test compound) and a labeled NAD⁺ (e.g., a colorimetrically-labeled, fluorescently-labeled, biotinylated-, or radioisotope-labeled NAD⁺). The amount of labeled ADP-ribose covalently attached to PARP-16 is subsequently measured. In a method for identifying a candidate compound that is a PARP-16-specific inhibitor, the compound mediates a decrease (e.g., at least a 5%, 10%, 15%, 20%, 25%, 30%, 35%, 40%, 45%, 50%, 55%, 60%, 65%, 70%, 80%, 90%, 95%, or even 100% decrease) in the amount of labeled ADP-ribose covalently attached to PARP-16 and/or another PARP-16 substrate (e.g., PERK and/or IRE1α). In a method for identifying a candidate compound that is a PARP-16-specific activator, the compound mediates an increase (e.g., at least a 5%, 10%, 15%, 20%, 25%, 30%, 35%, 40%, 45%, 50%, 55%, 60%, 65%, 70%, 80%, 90%, 95%, or even 100% increase) in the amount of labeled ADP-ribose covalently attached to PARP-16 and/or another PARP-16 substrate (e.g., PERK and/or IRE1α).

The PARP-16 utilized in each assay may be purified, partially purified (e.g., at least 30% pure, at least 40% pure, at least 50% pure, at least 60% pure, at least 70% pure, at least 80% pure, at least 85% pure) or may be present in a cell lysate (e.g., a bacterial cell lysate, a yeast cell lysate, or a mammalian cell lysate), in a biological fluid from a transgenic animal (e.g., milk or serum), or an extracellular medium. The PARP-16 protein utilized in the assay may be bound to substrate, such as, but not limited to, a solid surface (e.g., a multi-well plate), a resin, or a bead (e.g., a magnetic bead).

In preferred assays, an activator or inhibitor that increases or decreases the amount of labeled ADP-ribose covalently attached to PARP-16 while having no or little (e.g., less than 50%, less than 40%, less than 30%, less than 25%, less than 20%, less than 15%, less than 10%, or less than 5% change (e.g., increase or decrease)) effect on the amount of labeled ADP-ribose covalently attached to other PARP fusion proteins, is identified as a PARP-16-specific activator or inhibitor, respectively. For example, the assay desirably identifies a compound that specifically inhibits the amount of labeled ADP-ribose covalently attached to PARP-16. Another assay desirably identifies an agent that specifically increases the amount of labeled ADP-ribose covalently attached to PARP-16.

A variety of different compounds may be tested in the above-described methods of the invention. For example, a tested compound may be a derived from or present in a crude lysate (e.g., a lysate from a mammalian cell or plant extract) or be derived from a commercially available chemical libraries. Large libraries of both natural product or synthetic (or semi-synthetic) extracts or chemical libraries are presently available and known in the art. The screening methods of the present invention are appropriate and useful for testing compounds from a variety of sources for activity as a PARP-16-specific activator or inhibitor. Compounds from commercial sources can be tested, as well as commercially available derivatives and analogs of identified PARP inhibitors or activators. In addition, the initial screens may be performed using a diverse library of compounds from various compound libraries. Such compound libraries can be combinatorial libraries, natural product libraries, or other small molecule libraries.

The synthesis of combinatorial libraries is well known in the art and has been reviewed (see, e.g., Gordon et al., J. Med. Chem. 37: 1385-1401, 1994; Hobbes et al, Acc. Chem. Res. 29: 114, 1996; Armstrong, et al., Acc. Chem. Res. 29: 123, 1996; Ellman Acc. Chem. Res. 29: 132, 1996; Gordon et al., Ace. Chem. Res. 29: 144, 1996; Lowe, Chem. Soc. Rev. 309, 1995; Blondelle et al., Trends Anal. Chem. 14: 83, 1995; Chen et al., J. Am. Chem. Soc. 116: 2661, 1994; U.S. Pat. Nos. 5,359,115, 5,362,899, and 5,288,514; PCT Publication Nos. WO1992/10092, WO1993/09668, WO1991/07087, WO1993/20242, and WO1994/08051).

Preferably, for PARP-16-specific inhibitors, combinatorial libraries of test compounds can be synthesized based upon known pan-PARP inhibitors to increase chances of identifying suitable candidate compounds useful for treating a subject with an ER stress-related condition. PARP inhibitors which may serve as structural scaffolds for the generation of combinatorial libraries include, but are not limited to, the compounds disclosed in PCT Publication Nos. WO2004/080976, WO1996/022791, and WO2008/063644, which are herein incorporated by reference in their entirety.

A candidate compound may be a protein, a peptide, a DNA, or a RNA aptamer (e.g., a RNAi molecule), a lipid, or a small molecule (e.g., a lipid, carbohydrate, a bioinorganic molecule, or an organic molecule).

Compounds that may be tested as a PARP-16-specific activator include nucleic acids that contain a sequence encoding one or more domains of the PARP-16 protein and proteins that may increase expression or activity of PARP-16 by post-translation modification (e.g., a kinase that phosphorylates PARP-16, thereby increasing its activity).

### Kits

The invention further provides kits for treating a subject with an ER stress-related condition. The kits therefore include a pharmaceutical composition that modulates PARP-16 expression or activity. For example, a kit may contain one or more PARP-16-specific inhibitors or activators.

### Methods of Diagnosing an ER Stress-Related Condition

On the basis of the identified role of PARP-16 in UPR activation, the present invention provides assays useful in the diagnosis of ER stress-related conditions, such as cancer and Alzheimer's disease. Accordingly, the diagnosis of ER stress-related conditions may be performed by measuring the level of expression or activity of PARP-16 in a sample taken from a subject. This level of activity can then be compared to a control sample, for example, a sample taken from a subject without an ER stress-related condition. Increased level of PARP-16 expression or activity, relative to the control, is taken as a diagnostic of an ER stress-related condition.

Analysis of levels of PARP-16 mRNA or polypeptide or activity of the peptide may be used as the basis for screening the subject sample (e.g., blood or tissue sample). Methods for screening polypeptide levels may include immunological techniques standard in the art (e.g., western blot or ELISA), or may be performed using chromatographic or other protein purification techniques. In another embodiment, the activity (e.g., the mono-ADP-ribosylating activity) of PARP-16 may be measured, where an increase in mono-ADP-ribosylated PARP substrates (e.g., PERK, IRE1α, and/or PARP-16) is diagnostic of an ER stress-related condition. Such activity may be measured by any standard method in the art (e.g., Western immunoblot, mass spectrometry).

### Methods of Treating an ER Stress-Related Condition by Modulation of PARP-16

The methods and kits of the invention can be used for treating a subject with an ER stress-related condition, such as cancer, a protein folding/misfolding disease, or a myelinating cell-related disease. In particular, the methods of the invention can be used to treat individuals with cancer, protein folding/misfolding disease, myelinating cell-related disease, bipolar disorder, diabetes mellitus, Wolcott-Rallison syndrome, ischemia/reperfusion injury, stroke, neurodegeneration, atherosclerosis, neoplasia, hypoxia, or hypoglycemia.

Preferably, methods that include administering a pharmaceutical composition that decreases PARP-16 expression or activity (e.g., a PARP-16-specific inhibitor) may be used to treat cancer, protein folding/misfolding disease, diabetes mellitus, Wolcott-Rallison syndrome, ischemia/reperfusion injury, stroke, neurodegeneration, atherosclerosis, neoplasia, hypoxia, or hypoglycemia in a subject. Methods that include administering a pharmaceutical composition that increases PARP-16 expression or activity (e.g., includes a PARP-16-specific activator) may be used to treat a myelinating cell-related disease, protein folding/misfolding disease, or bipolar disorder in a subject.

The cancer may be colon adenocarcinoma, esophagus adenocarcinoma, liver hepatocellular carcinoma, squamous cell carcinoma, pancreas adenocarcinoma, islet cell tumor, rectum adenocarcinoma, gastrointestinal stromal tumor, stomach adenocarcinoma, adrenal cortical carcinoma, follicular carcinoma, papillary carcinoma, breast cancer, ductal carcinoma, lobular carcinoma, intraductal carcinoma, mucinous carcinoma, phyllodes tumor, Ewing's sarcoma, ovarian adenocarcinoma, endometrium adenocarcinoma, granulose cell tumor, mucinous cystadenocarcinoma, cervix adenocarcinoma, vulva squamous cell carcinoma, basal cell carcinoma, prostate adenocarcinoma, giant cell tumor of bone, bone osteosarcoma, larynx carcinoma, lung adenocarcinoma, kidney carcinoma, urinary bladder carcinoma, Wilm's tumor, or lymphoma.

The protein folding/misfolding disease may be Alzheimer's disease, Parkinson's disease, amyotrophic lateral sclerosis (ALS), Creutzfeldt-Jakob disease, bovine spongiform encephalopathy (BSE), light chain amyloidosis (AL), Huntington's disease, spinobulbar muscular atrophy (Kennedy disease), Machado-Joseph disease, dentatorubral-pallidoluysian atrophy (Haw River Syndrome), or spinocerebellar ataxia.

The myelinating cell-related disease may be multiple sclerosis (MS), Charcot-Marie-Tooth disease (CMT), Pelizaeus-Merzbacher Disease (PMD), or Vanishing White Matter Disease (VWMD).

Depending on the stage of the protein folding/misfolding disease of a subject, the subject may be preferably treated by administering a pharmaceutical composition that decreases PARP-16 expression or activity (e.g., includes a PARP-16-specific inhibitor). In other instances, the subject having the protein folding/misfolding disease may be preferably treated by administering a pharmaceutical composition that increases PARP-16 expression or activity (e.g., includes a PARP-16-specific activator). Whether administration of a PARP-16-inhibiting composition or PARP-16-activating composition is preferable may be determined by a physician, for example. Preferably, pharmaceutical compositions that increase PARP-16 expression or activity may be administered to the subject if the protein aggregation is expected to be resolved by activation of the UPR or if treatment is prophylactic. Alternatively, if the predicted levels of protein aggregation are not likely to be resolved by activation of the UPR and/or if activation of the UPR is likely to trigger unwanted cellular apoptosis, pharmaceutical compositions that decrease PARP-16 expression or activity may be preferred.

### Pharmaceutical Formulation and Administration of the Compositions of the Invention

### Administration

The methods of the invention include administering to a subject (e.g., a human) a therapeutically effective amount of a pharmaceutical composition that decreases or increases PARP-16 expression to treat, prevent, ameliorate, inhibit the progression of, or reduce the severity of one or more symptoms of an ER stress-related condition (e.g., cancer) in the subject. Examples of the symptoms of, e.g., cancer that can be treated using the compositions of the invention include, e.g., fatigue, weight change, skin change, persistent coughing, changes in bowel or bladder habits, difficulty swallowing, hoarseness, persistent indigestion after eating, persistent and unexplained muscle or joint pain, fever, headache, chills, diarrhea, vomiting, rash, dizziness, seizures, organ failure, personality changes, confusion. These symptoms, and their resolution during treatment, may be measured by, e.g., a physician during a physical examination or by other tests and methods known in the art.

The pharmaceutical compositions utilized in the methods described herein can be formulated for administration by a route selected from, e.g., parenteral, dermal, transdermal, ocular, inhalation, buccal, sublingual, perilingual, nasal, rectal, topical administration, and oral administration. Parenteral administration includes intravenous, intraperitoneal, subcutaneous, and intramuscular administration. Parenteral, intranasal, or intraocular administration may be provided by using, e.g., aqueous suspensions, isotonic saline solutions, sterile and injectable solutions containing pharmacologically compatible dispersants and/or solubilizers, for example, propylene glycol or polyethylene glycol, lyophilized powder formulations, and gel formulations. The preferred method of administration can vary depending on various factors (e.g., the components of the composition being administered and the severity of the condition being treated). Formulations suitable for oral or nasal administration may consist of liquid solutions, such as an effective amount of the composition dissolved in a diluent (e.g., water, saline, or PEG-400), capsules, sachets, tablets, or gels, each containing a predetermined amount of the composition of the invention. The pharmaceutical composition may also be an aerosol formulation for inhalation, e.g., to the bronchial passageways. Aerosol formulations may be mixed with pressurized, pharmaceutically acceptable propellants (e.g., dichlorodifluoromethane, propane, or nitrogen). In particular, administration by inhalation can be accomplished by using, e.g., an aerosol containing sorbitan trioleate or oleic acid, for example, together with trichlorofluoromethane, dichlorofluoromethane, dichlorotetrafluoroethane, or any other biologically compatible propellant gas.

In some instances, the compositions of the methods of the invention may be significantly effective if co-administered with an immunostimulatory agent or adjuvant. Suitable adjuvants well-known to those skilled in the art include, e.g., aluminum phosphate, aluminum hydroxide, QS21, Quil A (and derivatives and components thereof), calcium phosphate, calcium hydroxide, zinc hydroxide, glycolipid analogs, octodecyl esters of an amino acid, muramyl dipeptides, polyphosphazene, lipoproteins, ISCOM matrix, DC-Chol, DDA, cytokines, and other adjuvants and derivatives thereof.

Pharmaceutical compositions according to the invention described herein may be formulated to release the composition immediately upon administration (e.g., targeted delivery) or at any predetermined time period after administration using controlled or extended release formulations. Administration of the pharmaceutical composition in controlled or extended release formulations is useful where the composition, either alone or in combination, has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window at the site of release; or (iii) a short biological half-life, so that frequent dosing during a day is required in order to sustain a therapeutic level.

Many strategies can be pursued to obtain controlled or extended release in which the rate of release outweighs the rate of metabolism of the pharmaceutical composition. For example, controlled release can be obtained by the appropriate selection of formulation parameters and ingredients, including, e.g., appropriate controlled release compositions and coatings. Suitable formulations are known to those of skill in the art. Examples include single or multiple unit tablet or capsule compositions, oil solutions, suspensions, emulsions, microcapsules, microspheres, nanoparticles, patches, and liposomes.

The pharmaceutical compositions of the methods of the invention may be administered to provide treatment to a subject having an ER stress-related condition, such as cancer. The compositions may be administered to the subject, e.g., 1, 2, 3, 4, 5, 6, 7, 8, 9, 10, 15, 20, 30, 35, 40, 45, 50, 55, or 60 minutes, 2, 4, 6, 10, 15, or 24 hours, 2, 3, 5, or 7 days, 2, 4, 6 or 8 weeks, 3, 4, 6, or 9 months, 1, 2, 3, 4, 5, 6, 7, 8, 9, 10, 15, 20 years or longer post-diagnosis of cancer.

When treating an ER stress-related condition (e.g., cancer, protein folding/misfolding disease), the pharmaceutical compositions of the methods of the invention may be administered to the subject either before the occurrence of symptoms or a definitive diagnosis or after diagnosis or symptoms become evident. For example, the compositions may be administered, e.g., immediately after diagnosis or the clinical recognition of symptoms or 2, 4, 6, 10, 15, or 24 hours, 2, 3, 5, or 7 days, 2, 4, 6 or 8 weeks, or even 3, 4, or 6 months after diagnosis or detection of symptoms.

The compositions may be sterilized by conventional sterilization techniques, or may be sterile filtered. The resulting aqueous solutions may be packaged for use as is, or lyophilized; the lyophilized preparation may be administered in powder form or combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of a PARP-16-modulating compound (e.g., a PARP-16-specific inhibitor or a PARP-16-specific activator) and, if desired, one or more agents, such as in a sealed package of tablets or capsules, or in a suitable dry powder inhaler (DPI) capable of administering one or more doses.

### Dosages

The dose or the number of treatments using the methods of the invention may be increased or decreased based on the severity of, occurrence of, or progression of, the ER stress-related condition in the subject (e.g., based on the severity of one or more symptoms of, e.g., cancer), but generally range from about 0.5 mg to about 3,000 mg of each compound per dose one or more times per week (e.g., 2, 3, 4, 5, 6, or 7 or more times per week).

The pharmaceutical compositions of the methods of the invention can be administered in a therapeutically effective amount that provides a protective effect against the ER stress-related condition (e.g., cancer). The dosage administered depends on the subject to be treated (e.g., the age, body weight, capacity of the immune system, and general health of the subject being treated), the form of administration (e.g., as a solid or liquid), the manner of administration (e.g., by injection, inhalation, dry powder propellant), and the cells targeted (e.g., myelinating cells, such as oligodendrocytes of the central nervous system or Schwann cells of the peripheral nervous system). The composition is preferably administered in an amount that provides a sufficient level of PARP-16-modulating compound that reduces or prevents one or more symptoms of, e.g., cancer, without undue adverse physiological effects in the subject caused by the treatment.

In addition, single or multiple administrations of the pharmaceutical compositions of the methods of the invention may be given to a subject with an ER stress-related condition (e.g., one administration or administration two or more times). Responsiveness of subjects treated by the compositions described herein may be measured by, e.g., a physician during a physical examination or by other tests and methods known in the art, e.g., by measuring tumor cell glucose uptake by fluorodeoxyglucose-positron emission tomography (FDG-PET). The dosages may then be adjusted or repeated as necessary.

A single dose of the pharmaceutical compositions of the methods of the invention may reduce, treat, or prevent one or more symptoms of the ER stress-related condition (e.g., cancer) in the subject. In addition, a single dose of the compositions can also be used to achieve therapy in subjects being treated for an ER stress-related condition. Multiple doses (e.g., 2, 3, 4, 5, or more doses) can also be administered, in necessary, to these subjects.

### Carriers, Excipients, Diluents

The methods of the invention include pharmaceutical compositions that decrease or increase PARP-16 expression or activity. Therapeutic formulations of the compositions are prepared using standard methods known in the art by mixing the active ingredient having the desired degree of purity with optional physiologically acceptable carriers, excipients, or stabilizers (Remington's Pharmaceutical Sciences, 21^{th} ed., A. Gennaro, 2005, Lippincott, Williams & Wilkins, Philadelphia, Pa.). Acceptable carriers, include saline, or buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagines, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG.

Optionally, but preferably, the formulation contains a pharmaceutically acceptable salt, preferably sodium chloride, and preferably at about physiological concentrations. Optionally, the formulations of the invention can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are preferred preservatives. Optionally, the formulations of the invention can include a pharmaceutically acceptable surfactant at a concentration of 0.005 to 0.02%.

### EXAMPLES

The following examples are to illustrate the invention. They are not meant to limit the invention in any way.

### Example 1

### PARP-16 is an Integral ER Membrane Protein

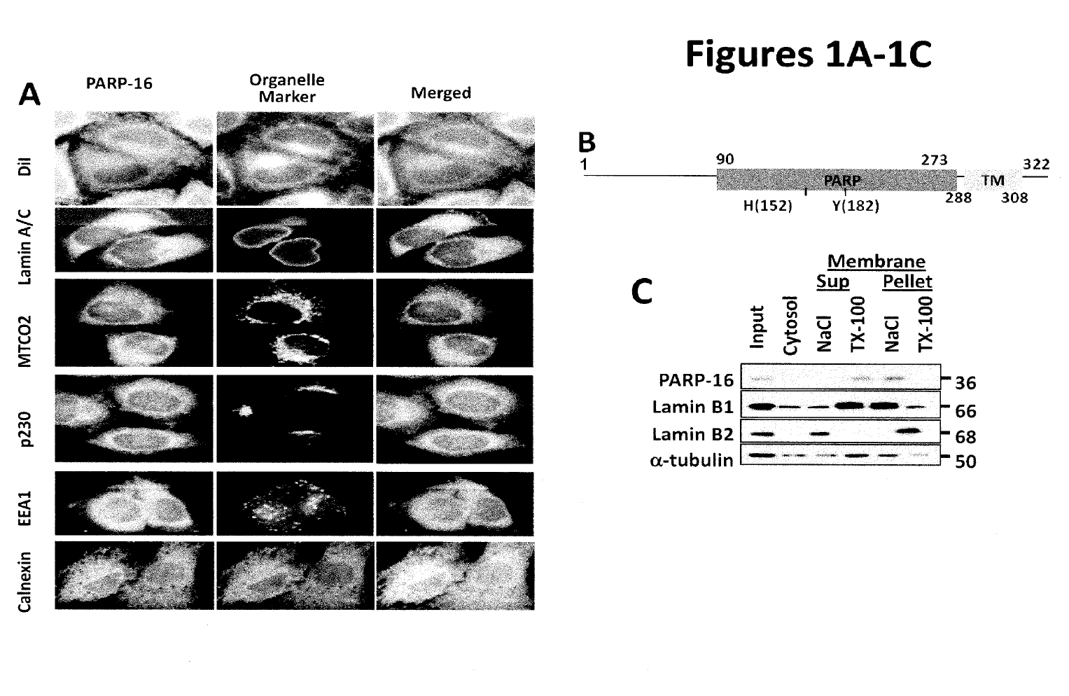

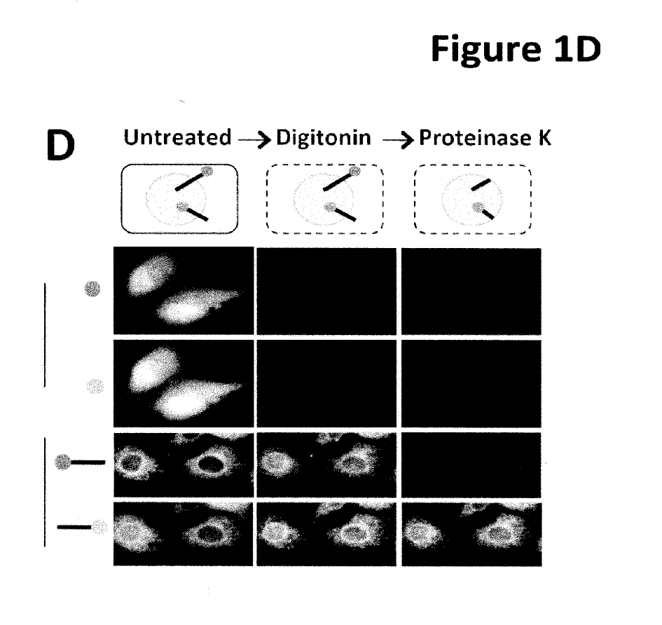

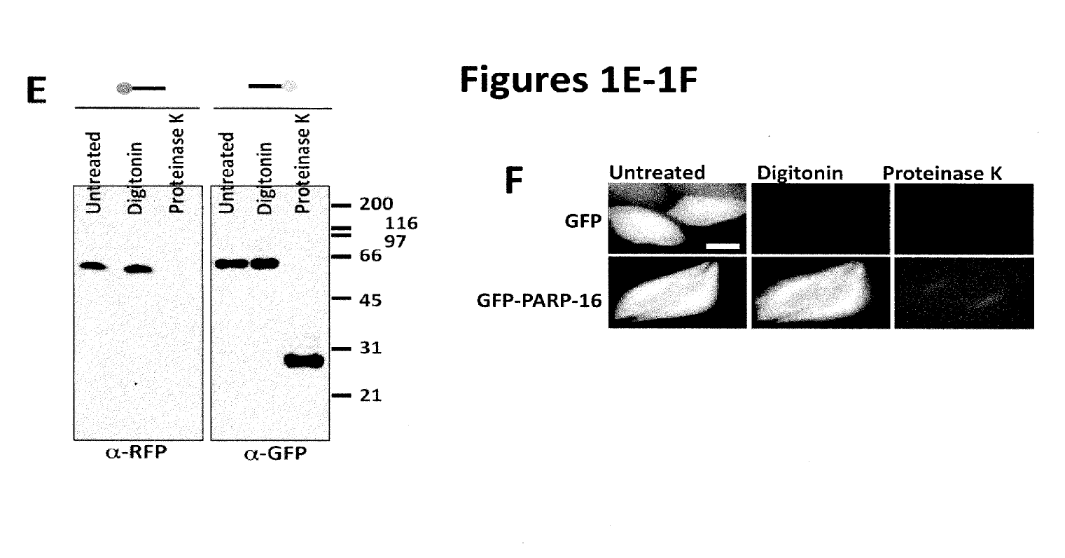

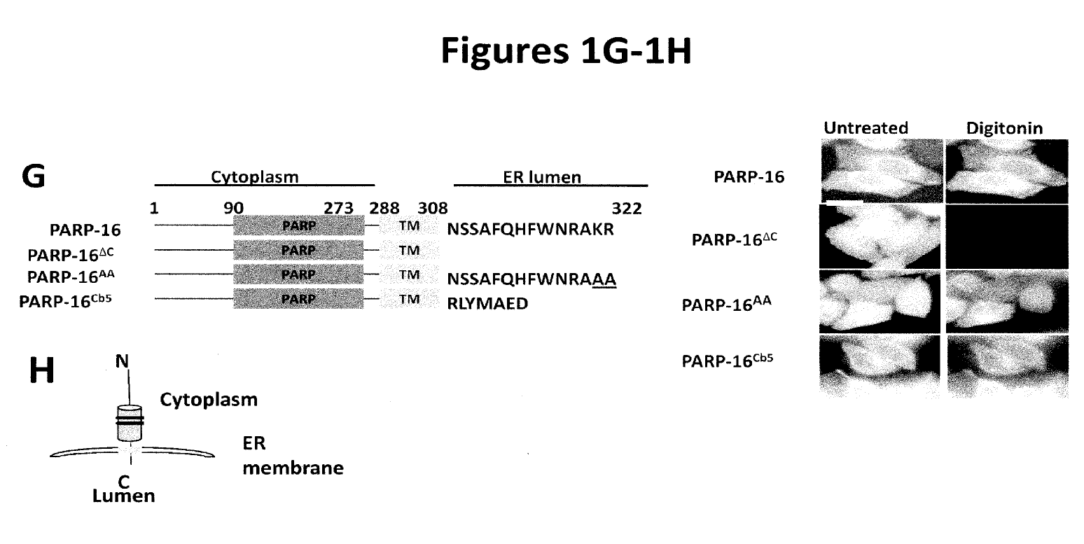

We previously identified a reticular membrane localization for uncharacterized PARP-16 in a screen analyzing PARP function using lypophilic dye DiI (FIG. 1A). To identify organelles to which it localizes, HeLa cells (utilized in all subsequent experiments) were stained with antibodies against PARP-16 and markers for membrane bound organelles—Calnexin, Lamin A/C, MTCO2, p230, and EEA1. Of these, PARP-16 and Calnexin localization strongly overlapped, suggesting that PARP-16 is an ER protein (FIG. 1A).

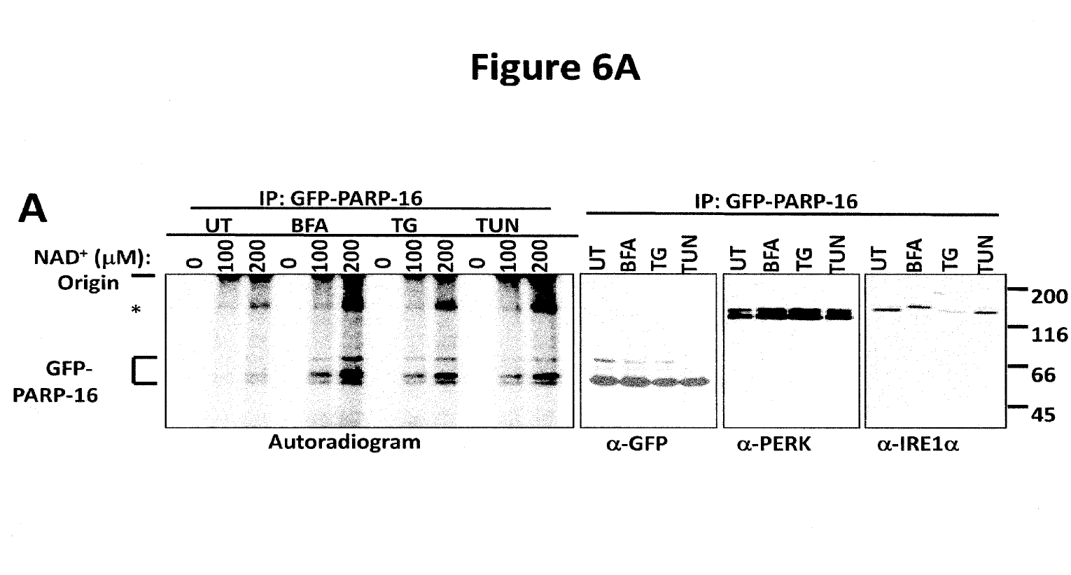

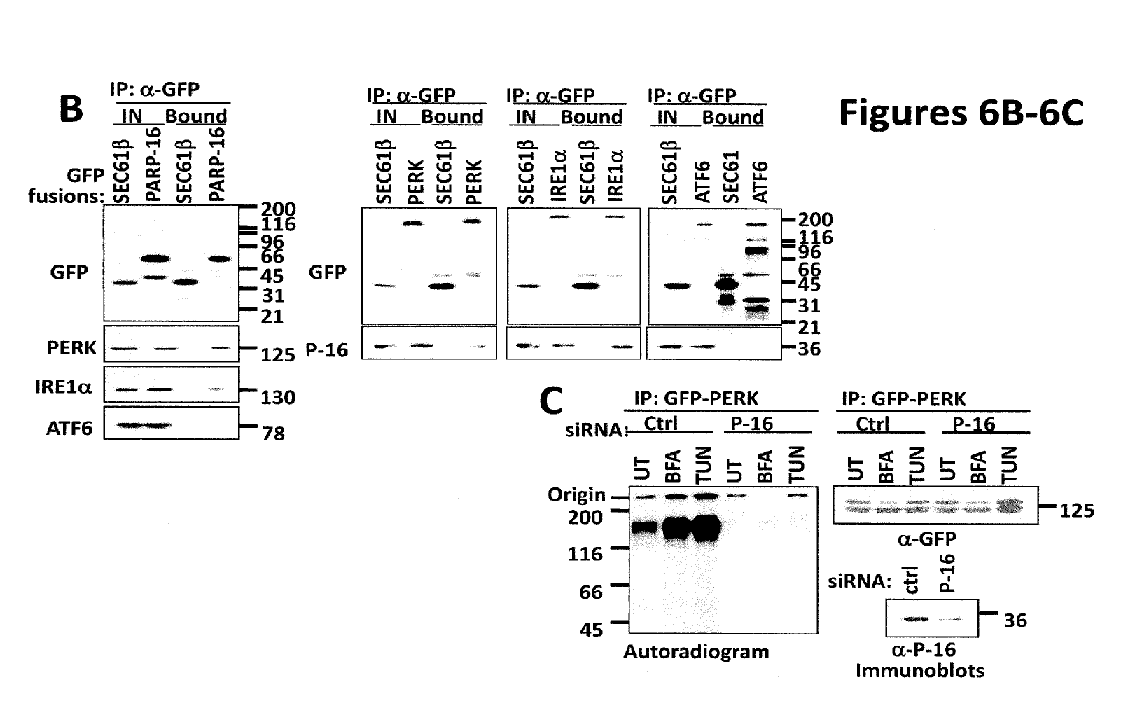

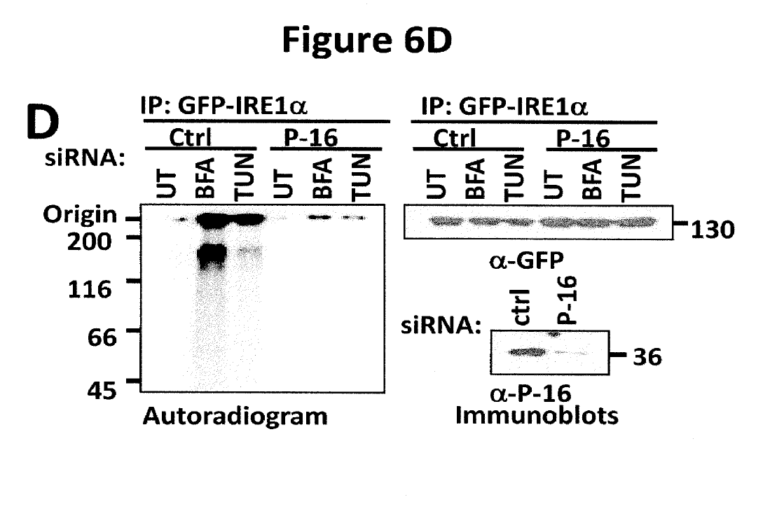

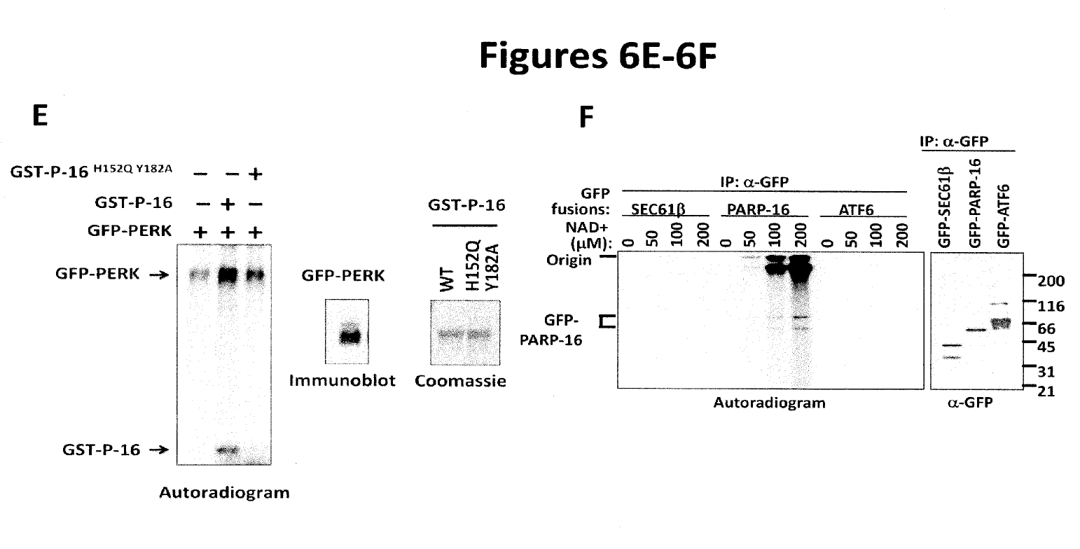

Based on primary sequence, PARP-16 is predicted to be a tail-anchored (TA) protein with a hydrophobic transmembrane domain at amino acid 288-308 (FIG. 1B; UniProtKB (Whitley et al., J. Biol. Chem. 271: 7583-7586, 1996)). TA proteins are single-spanning transmembrane proteins that contain cytoplasmic N-termini, short transmembrane domains (<30 a.a.) and C-terminal domains called C-tails (˜10-15 amino acids) positioned within the lumen of target organelles. C-tails target to the ER via net positive charge rather than specific amino acid composition and are inserted post-translationally into ER membrane via the GET (Golgi-ER trafficking) complex (Schuldiner et al., Cell 134: 634-645, 2008; Borgese et al., Curr. Opin. Cell Biol. 19: 368-375, 2007). To determine if PARP-16 is a TA protein, we performed membrane extraction assays to confirm that PARP-16 is transmembrane, protease protection assays to determine if the N-terminus is cytoplasmic, and truncation/mutation assays to determine if the C-terminus acts as a C-tail (Lorenz et al., Nat. Methods 3: 205-210, 2005). Treatment of purified membrane fractions with 1M NaCl released peripherally associated membrane protein Lamin B2 but not PARP-16, while treatment with 1% Triton X-100 resulted in the release of transmembrane protein Lamin B1 and PARP-16, identifying PARP-16 as a transmembrane protein (FIG. 1C). We next set out to determine the orientation of PARP-16 relative to the ER membrane using fluorescence protease protection assays (Lorenz et al., Nat. Methods 3: 205-210, 2005) in HeLa cells co-expressing N-terminal mCherry fusions of PARP-16 (mCherry-PARP-16) and C-terminal GFP fusions of PARP-16 (PARP-16-GFP). Control cells expressing mCherry and GFP lost detectable fluorescence signal upon plasma membrane permeabilization using digitonin (FIG. 1D). In contrast, mCherry-PARP-16 and PARP-16-GFP fluorescence was maintained upon digitonin treatment, confirming that PARP-16 is an integral membrane protein (FIG. 1D). Subsequent treatment with Proteinase K resulted in the loss of fluorescence from mCherry-PARP-16 but not PARP-16-GFP, suggesting that the C-terminally fused GFP was protected from Proteinase K activity by the ER membrane (FIG. 1D). Thus, the N-terminus of PARP-16 is present in the cytoplasm and the C-terminus is located inside the ER lumen. Bioinformatic sequence alignment predicts that the catalytic PARP domain of PARP-16 is located between amino acids 90-273 (FIG. 1B). To position the catalytic domain of PARP-16 relative to the ER membrane, we analyzed the electrophoretic mobility of the above proteins after membrane extraction. The molecular weight of mCherry-PARP-16 and PARP-16-GFP was not altered by digitonin treatment. Upon Proteinase K treatment, PARP-16-GFP detected by anti-GFP antibodies, resolved at a lower molecular weight and mCherry-PARP-16 was undetectable by anti-RPF antibodies due to loss of the proteolyzed N-terminal mCherry (FIG. 1E). In contrast, under this condition, PARP-16-GFP was detected at a lower molecular weight by anti-GFP antibodies, which predicts the catalytic domain of PARP-16 to be cytosolic and the transmembrane domain to be C-terminal to the catalytic domain. A N-terminal GFP-fusion to PARP-16, GFP-PARP-16 remained membrane associated in response to Digitonin treatment (in contrast to GFP only controls), while subsequent Proteinase K treatment resulted in loss of fluorescence, suggesting that the N-terminus or PARP-16 is cytoplasmic (FIG. 1F). Finally, a C-tail truncation (PARP-16^{ΔC}) and a PARP-16^{AA} mutant failed to localize to the ER (a small portion of PARP-16^{AA} remained ER associated) while a Cytochrome b5 chimera (PARP-16^{Cb5}) with PARP-16 C-tail replaced with ER-associated Cytochrome b5 C-tail retained ER localization even upon Digitonin treatment, demonstrating that the C-terminus of PARP-16 functions as a C-tail (FIGS. 1G and 1H). Consistent with this observation, our in vitro assay data demonstrate the catalytic activity on the cytoplasmic side of purified ER microsomes (FIGS. 6A and 6C).

### Example 2

### Characterization of PARP-16 Enzymatic Activity

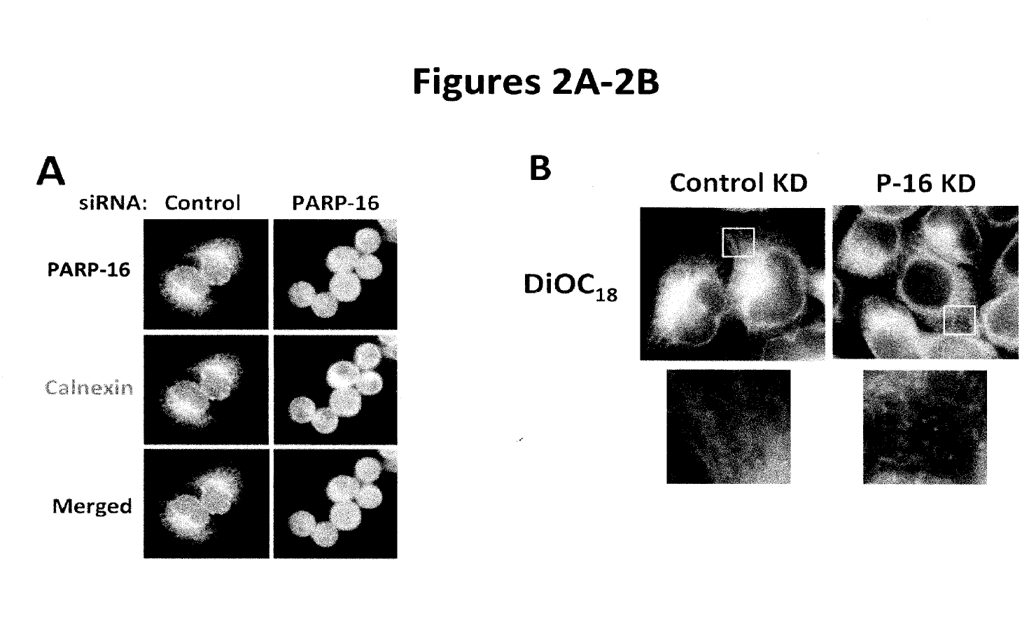

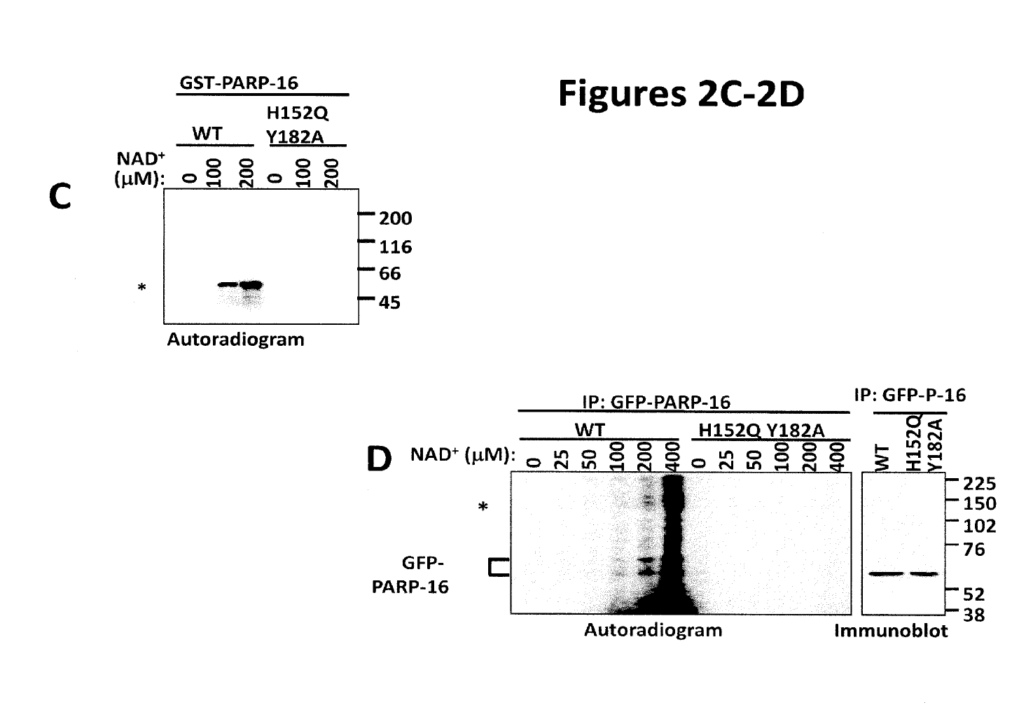

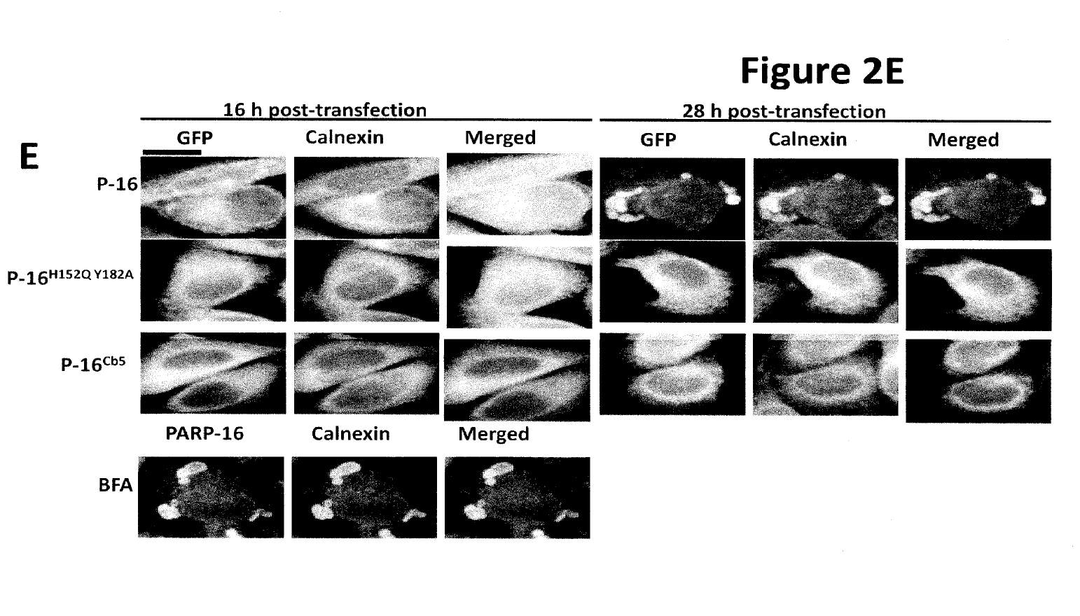

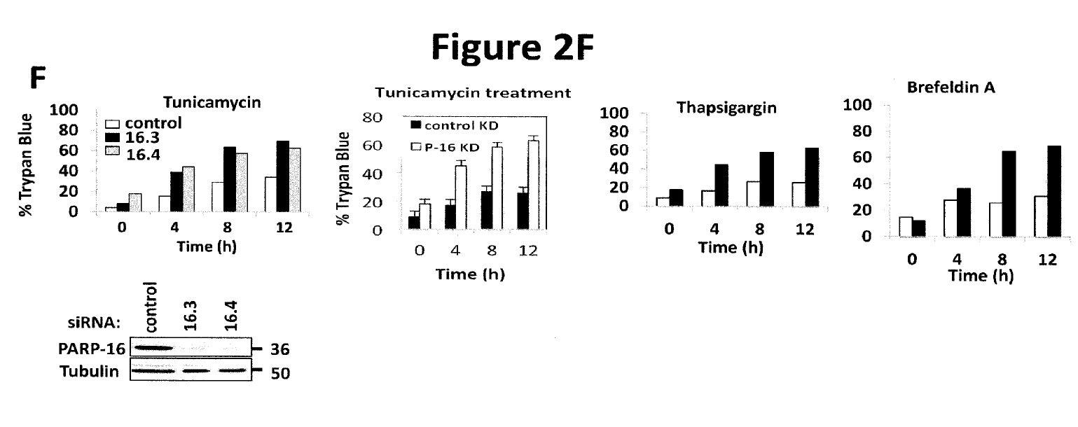

To identify physiological functions of PARP-16, we analyzed phenotypes of PARP-16 knockdown and over-expression in HeLa cells. As we previously reported, at least two sets of siRNAs against PARP-16 caused a dramatic change in cell morphology, resulting in round cells (FIG. 2A). FACS analysis did not show an increase in the G2/M DNA peak, suggesting that the phenotype is not a result of cell cycle defects. Instead, we observed a dramatic decrease in total ER as demonstrated by Calnexin staining (FIG. 2A). We further examined ER structure in the PARP-16 knockdown by examining intracellular membranes using lipophilic dye DiOC₁₈ (FIG. 2B). In control knockdown cells, reticular and vesicular structures were observed in the cytoplasm. In contrast, in PARP-16 knockdown cells, the reticular structures were greatly reduced; instead, numerous puncta were observed. These results suggest that PARP-16 is required for the reticular organization of the ER.

Human PARP-16 (ADP-ribosyl)ates itself and contains histidine and tyrosine residues at amino acid 152 and 182 within its catalytic domain, residues thought to be critical for enzymatic activity (FIG. 1B) (Kleine et al., Mol. Cell 32: 57-69, 2008). To determine if these residues are required for enzymatic activity, GST-PARP-16 and GST-PARP-16^{H152Q Y182A} were expressed and purified in E. coli, and ³²P-NAD⁺ incorporation assays performed. Self-modification of GST-PARP-16 was detected at its molecular weight in a NAD⁺ dose-dependent manner, while GST-PARP-16^{H152Q Y182A} exhibited incorporation activity at ˜6% of wild-type (FIG. 2C).

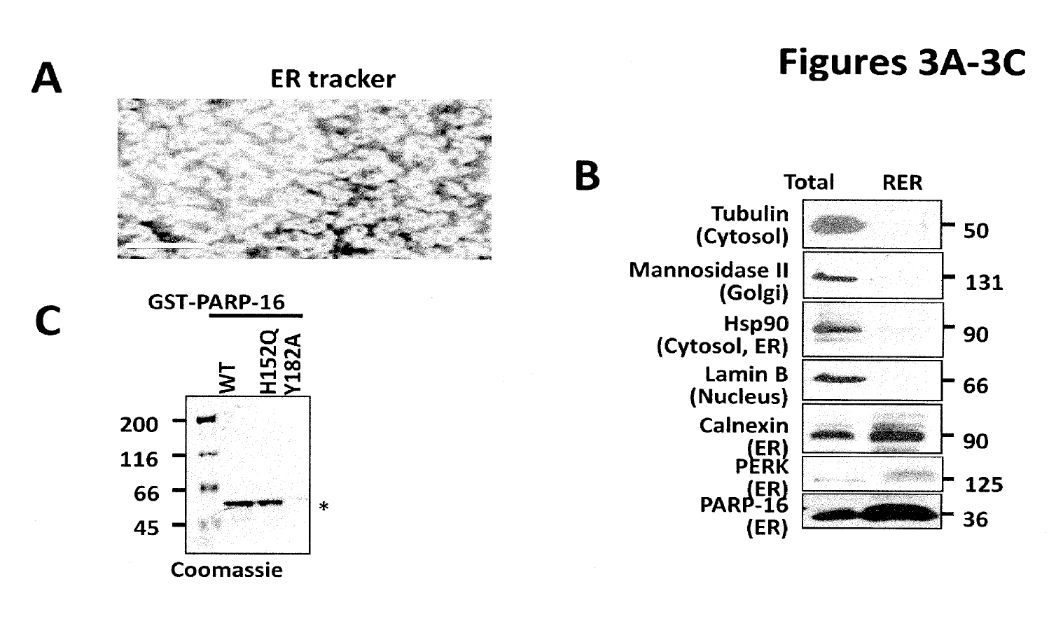

Analysis of PARP-16 membrane topology suggests its catalytic domain is cytoplasmic (FIG. 1H). To determine if PARP-16 ADP-ribosylation activity is cytoplasmic, and examine its function in the context of ER membrane, we developed an ER microsome assay to monitor NAD⁺ incorporation called the ER microsome (ADP-ribosyl)ation Assay (EMAA). Microsomes were purified from cells expressing GFP-PARP-16 or GFP-PARP-16^{H152Q Y182A}, incubated with ³²P-NAD⁺, dissolved to extract and purify GFP-PARP-16, then ³²P-NAD⁺ incorporation into GFP-PARP-16 assayed via autoradiography (Stephens et al., Methods Mol Biol. 419: 197-214, 2008). Microsomes purified for this purpose stained positive for ER tracker, were highly enriched in ER proteins, and were intact since they did not contain protein from other cellular compartments (FIGS. 3A and 3B). Since intact ER microsomes are impermeable to NAD⁺, any incorporation of ³²P-NAD⁺ occurs outside of the microsome lumen (Hamman et al., Cell 89: 535-544, 1997). Self-modification of GFP-PARP-16 was detected at its molecular weight in a NAD⁺ dose-dependent manner while GFP-PARP-16^{H152Q Y182A} failed to incorporate NAD⁺ (FIG. 2B, left), suggesting that PARP-16 (ADP-ribosyl)ation activity is cytoplasmic and requires His152 and Tyr182. Multiple migrating forms of GFP-PARP-16 were detected. In addition, ³²P-NAD⁺ was incorporated at higher molecular weight, suggesting the presence of binding proteins that are modified by PARP-16 (FIG. 2D, asterisk).

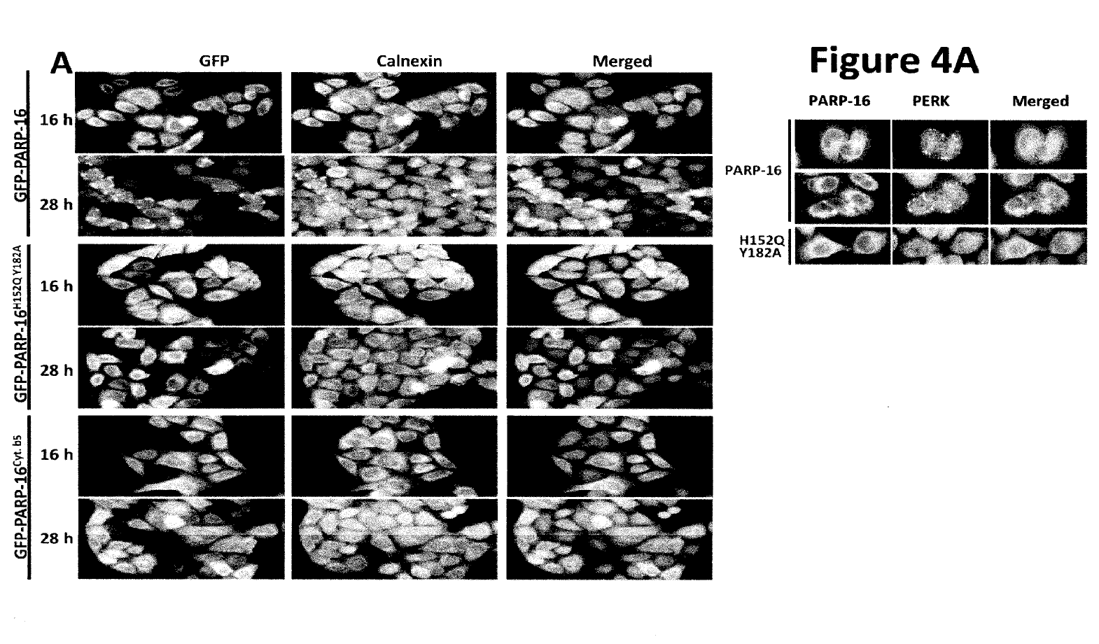

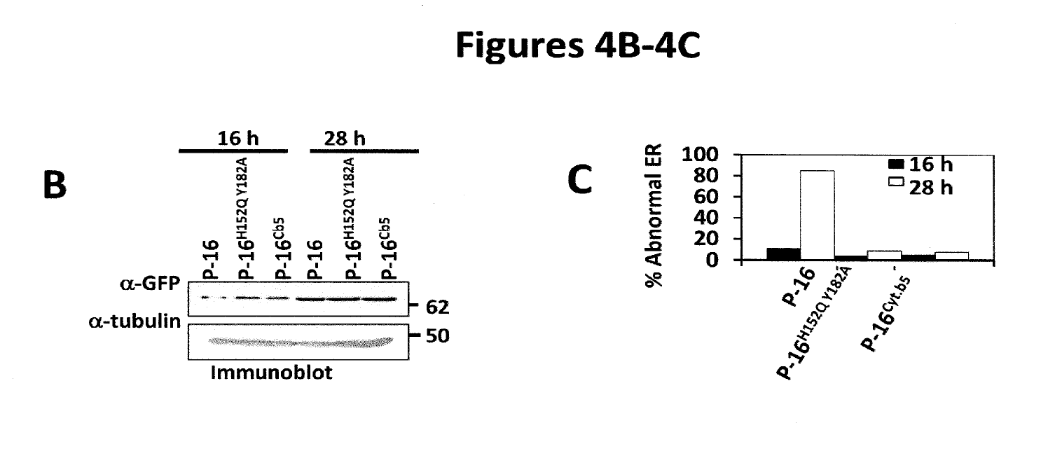

Interestingly, prolonged PARP-16 over-expression (>28 h) resulted in abnormal ER morphology with >80% of PARP-16 overexpressing cells containing abnormal globular ER structures (FIGS. 2E and 4A-4C). This phenotype was time and/or protein concentration dependent as ER appeared normal at 16 h of expression, and required PARP-16 enzymatic activity and an intact C-tail as only ˜5% of cells expressing PARP-16^{H152Q Y182A} or PARP-16^{Cb5} at levels similar to wild type PARP-16, contained abnormal ER (FIGS. 2E and 4A-4C). Since GFP-PARP-16 and GFP-PARP-16^{H152Q Y182A} both localized to the ER, enzymatic activity is not required for ER localization.

Abnormal ER structures resulting from prolonged PARP-16 over-expression resemble ER from stressed cells (Sriburi et al., J. Cell Biol. 167: 35-41, 2004), leading us to examine PARP-16 function in the unfolded protein response (UPR) (FIG. 2E). The UPR is an ER stress response activated by an increase in unfolded proteins within the ER lumen. In mammals, three transmembrane ER stress sensors, PERK and IRE1α, kinases with functionally interchangeable luminal domains, and the transcription factor ATF6, regulate separate but interconnected UPR signaling pathways (Harding et al., Annu. Rev. Cell Dev. Biol. 18: 575-599, 2002; Kim et al., Nat. Rev. Drug Discov. 7: 1013-1030, 2008; Malhotra et al., Semin. Cell Dev. Biol. 18: 716-731, 2007; Ron et al., Nat. Rev. Mol. Cell Biol. 8: 519-529, 2007; Walter et al., Science 334: 1081-1086, 2011). Under non-stress conditions, each sensor is bound to and inhibited by BiP, an ER specific chaperone. To determine if PARP-16 functions in the UPR, we knocked it down with two siRNAs generated against distinct sequences, then activated the UPR using Tunicamycin, Brefeldin A, and Thapsigargin, and examined the effects. Consistent with perturbed UPR function, PARP-16 knock-down rendered cells highly sensitive to ER stress, resulting in increased cell death (FIG. 2F).

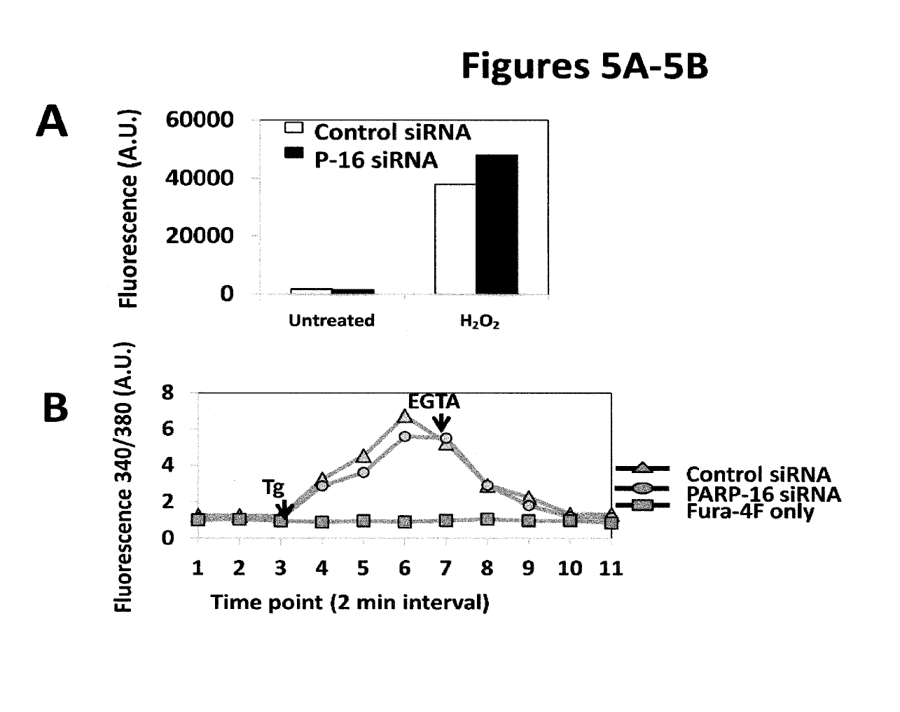

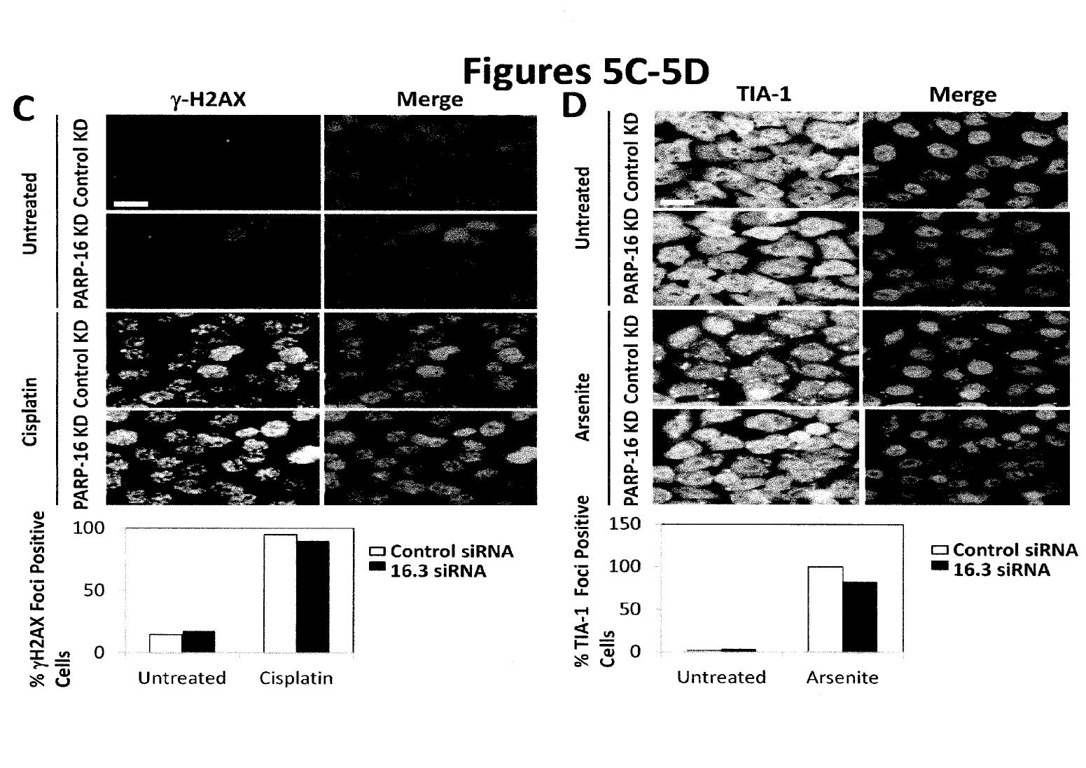

The high sensitivity to ER stress in PARP-16 knock-downs could also be explained by ER dysfunction or a general misregulation of cellular stress responses. However, ER function was intact in PARP-16 knock-downs as measured by intracellular concentration of ROS (reactive oxygen species) and Ca²⁺. PARP-16 and control knock-down cells exhibited similar ROS generation, measured via CM-H₂DCFDA in the presence or absence of H₂O₂, and similar Ca²⁺ leakage to the cytoplasm assayed via Fura-4F 340/380 nm fluorescence upon Thapsigargin treatment (FIGS. 5A and 5B). Non-UPR related cellular stress responses, such as DNA damage repair and the cytoplasmic stress response, both known to require PARP activity, were also intact in PARP-16 knock-downs. PARP-16 and control knock-down cells exhibited a similar response to DNA damage induced by Cisplatin, with similar levels of γ-H2AX foci formation, and cytoplasmic stress induced by Arsenite, although the number of cells positive for TIA-1 staining stress granules were slightly reduced relative to controls (FIGS. 5C and 5D).

### Example 3

### PARP-16 Enzymatic Activity is Important for the UPR

All known PARP-dependent stress responses result in up-regulation of PARP enzymatic activity (Hassa et al. Front. Biosci. 13: 3046-3082, 2008; Leung et al., Mol. Cell 42: 489-499, 2011). We examined PARP-16 enzymatic activity during the UPR via EMAA using cells expressing GFP-PARP-16 for 16 hours, a condition that did not affect ER organization (FIG. 2E). All subsequent EMAAs were performed in this manner. Cells expressing GFP-PARP-16 were treated +/−ER stress inducing agents, and PARP-16 activity assayed. ER stress resulted in significant increases in GFP-PARP-16 self-modification in a NAD⁺ dose-dependent manner (5-8 fold increase at 100 μM NAD⁺ and 8-13 fold increase at 200 μM NAD⁺, depending on stressor), and a dramatic electrophoretic mobility shift of GFP-PARP-16 was detected via immunoblot and autoradiogram (FIG. 6A). Additional higher molecular weight bands were also observed on the autoradiogram, migrating at the molecular weight of PERK (125 kD) and IRE1α (130 kD), but not ATF6 (75 kD). PERK and IRE1α were found in these GFP-PARP-16 precipitates via immunoblot under 450 mM NaCl conditions, demonstrating a robust association between PARP-16, PERK and IRE1α (FIG. 6A, right panels). These high molecular weight bands of NAD⁺ incorporation could represent (ADP-ribosyl)ation of PERK and IRE1α.

To determine if PARP-16 binds to ER stress sensors in the absence of ER stress, GFP fusions to PARP-16, PERK, IRE1α, ATF6 or SEC61β, an UPR-unrelated ER transmembrane protein, were expressed and co-immunoprecipitation assays performed. While PERK and IRE1α were present in GFP-PARP-16 precipitates, and PARP-16 was identified in GFP-PERK and IRE1α precipitates, no significant binding was identified between ATF6, SEC61β, and PARP-16 FIG. 6B). Thus, PARP-16 selectively binds to PERK and IRE1α but not ATF6 in the presence or absence of ER stress.

### Example 4

### PARP-16 is Required for PERK and IRE1α Regulation

Our data suggested that PERK and IRE1α could be substrates of PARP-16. We examined NAD⁺ incorporation onto GFP-PERK or GFP-IRE1α via EMAA in cells transfected with control or PARP-16 siRNA, treated with or without ER stress inducing drugs. Low level (ADP-ribosyl)ation of GFP-PERK and GFP-IRE1α was detected in control knock-down cells in the absence of drug (FIGS. 6C and 6D), likely due to the previously described UPR induction upon PERK or IRE1α expression (Bertolotti et al., Nat. Cell Biol. 2: 326-332, 2000; Kimata et al., Curr. Opin. Cell Biol. 23: 135-142, 2011). (ADP-ribosylation of GFP-PERK and GFP-IRE 1α increased under ER stress (5 fold and 4-11 fold, respectively, with differences dependent on stressor). In both cases this increase required PARP-16, as modification was dramatically reduced in PARP-16 knock downs (FIGS. 6C and 6D). In addition, recombinant GST-PARP-16, but not GST-PARP-16^{H152Q Y182A}, ADP-ribosylated PERK purified from ER microsomes of unstressed PARP-16 knockdown cells (FIG. 6E). Neither GFP-SEC61β nor GFP-ATF6 were (ADP-ribosyl)ated in similar assays (FIG. 6F).

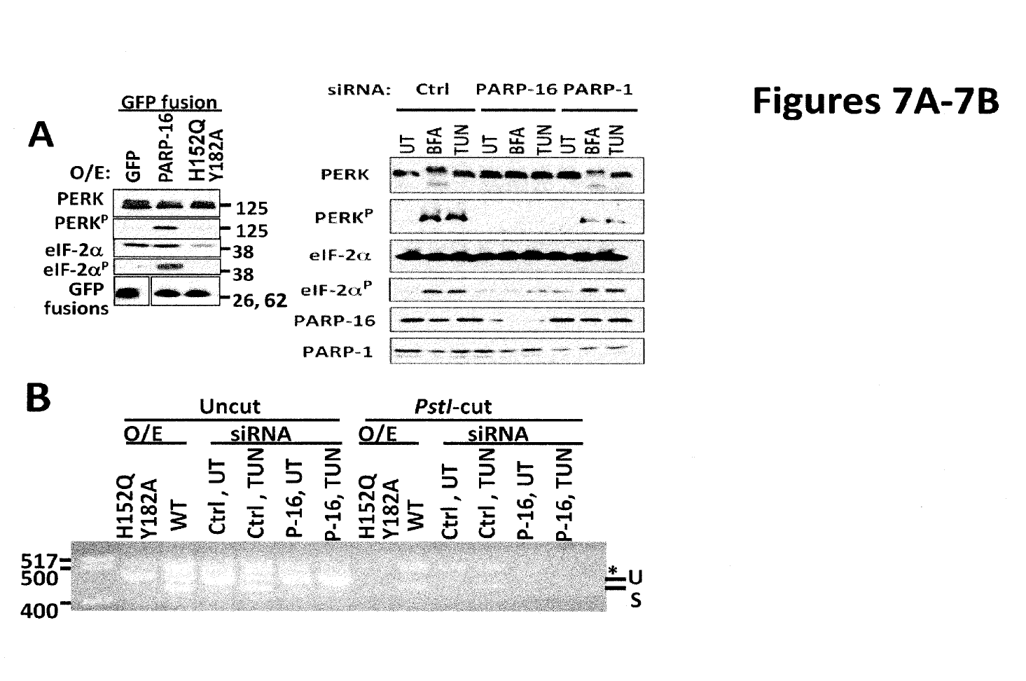

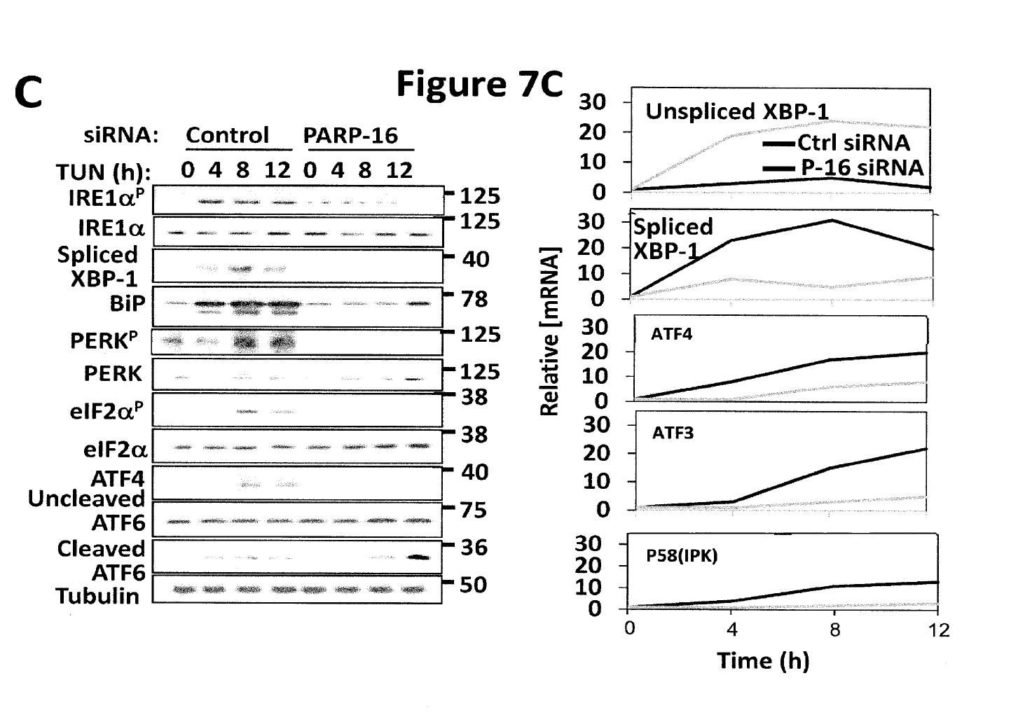

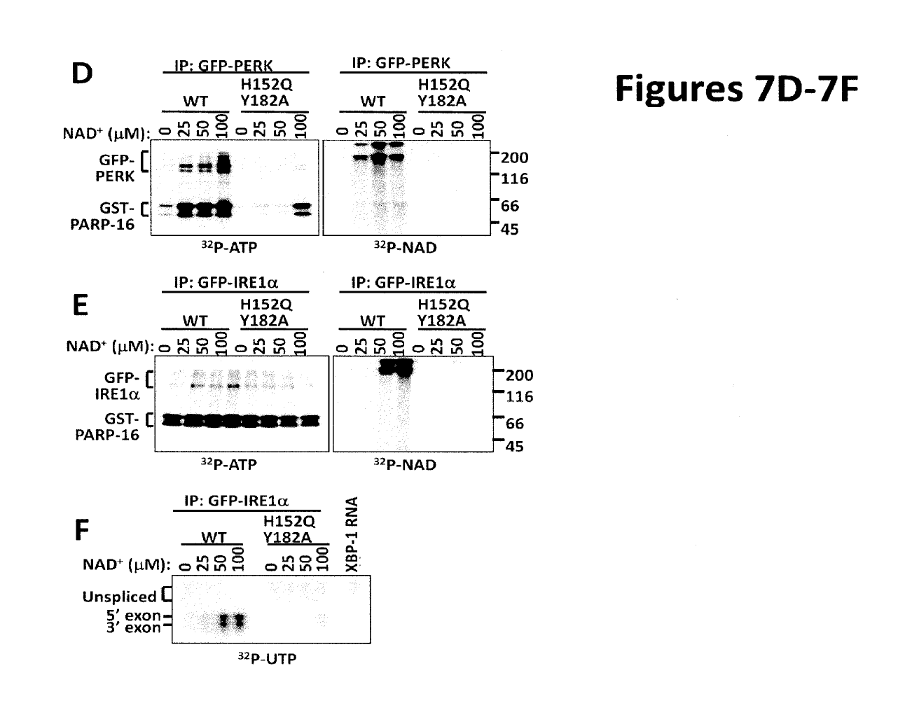

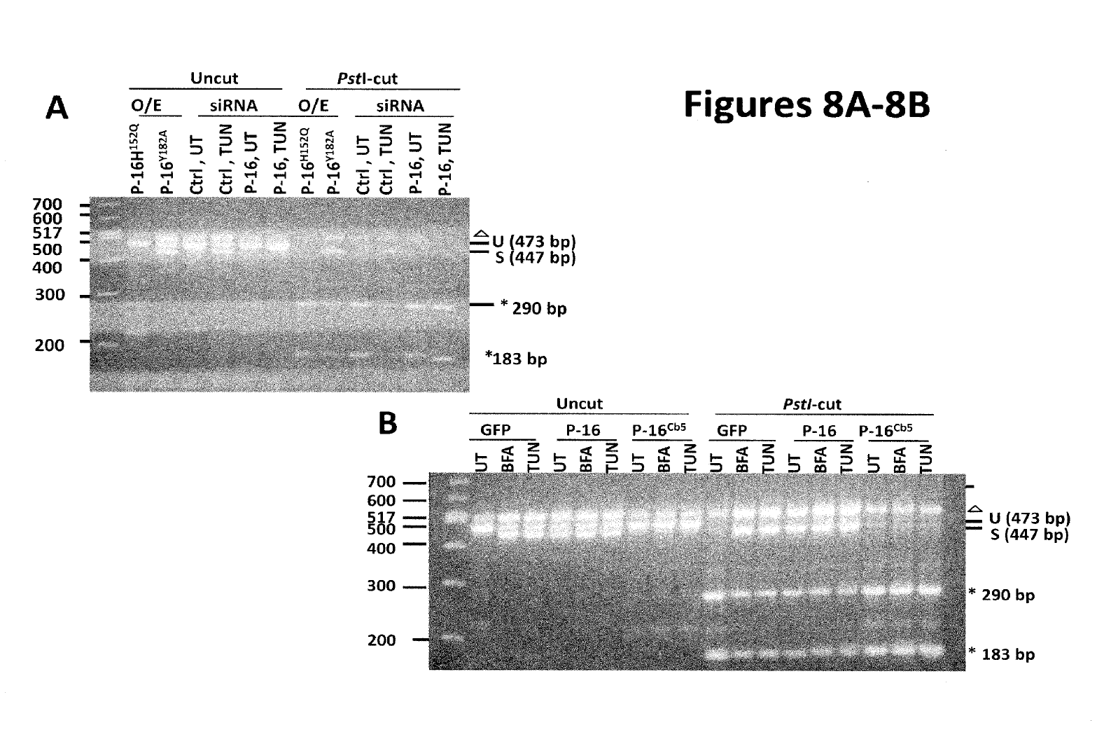

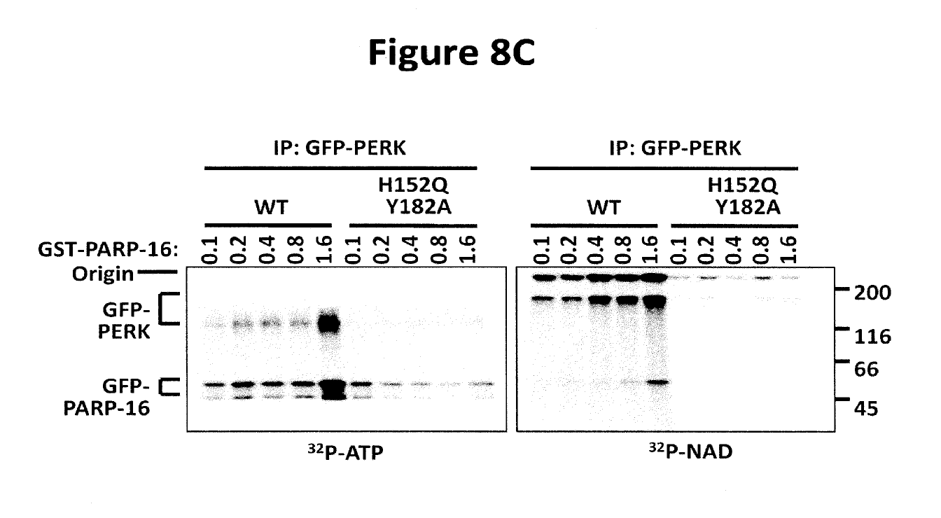

To determine the effects of (ADP-ribosyl)ation on PERK and IRE1α signaling, GFP-PARP-16, GFP-PARP-16^{H152Q Y182A}, or GFP alone were over-expressed at similar concentrations, and PERK and IRE1α activation examined using two standard assays; (i) detection of PERK phosphorylation at Thr 981 and phosphorylation of its substrate eIF2α at Ser 51 using phospho-specific antibodies, and (ii) monitoring splicing of the IRE1α substrate XBP-1 mRNA. Over-expression of GFP-PARP-16, but not GFP-PARP-16^{H152Q Y182A} or GFP resulted in PERK and eIF2α phosphorylation and XBP-1 splicing (FIGS. 7A, 7B, and 8A), suggesting that (ADP-ribosyl)ation by PARP-16 is sufficient to activate PERK and IRE1α.

To determine if PARP-16 is required for PERK or IRE1α activation, we compared activation in PARP-16 knock-downs to controls. Control cells treated with Brefeldin A or Tunicamycin resulted in robust phosphorylation of PERK and eIF2α, and XBP-1 splicing, while PARP-16 knock-downs similarly treated failed to activate PERK or IRE1α (FIGS. 7A, 7B, and 8A). Since PERK and IRE1α activity result in the time-dependent activation of downstream transcriptional programs regulated by ATF4 and spliced XBP-1, respectively, we analyzed PERK and IRE1α signaling every 4 hours over a 12-hour period in PARP-16 knock-downs and controls treated with Tunicamycin. Components of each pathway were analyzed via immunoblot or RT-qPCR analysis. While IRE1α activation, detected by phosphorylation of IRE1α, occurred 4 hours post-treatment in controls, such phosphorylation was barely detectable in PARP-16 knock-downs at any time (FIG. 7C, left). At 4 hours, spliced XBP-1 protein began to accumulate in controls, but was undetectable in PARP-16 knock-downs (FIG. 7C, left). IRE1α-dependent transcriptional programs were also defective in PARP-16 knock-downs. In control cells, unspliced XBP-1 mRNA decreased and spliced XBP-1 mRNA increased (FIG. 7C, right), whereas in PARP-16 knock-downs, unspliced XBP-1 mRNA increased, due to ATF6 activation, and spliced XBP-1 mRNA induction was reduced 5-fold at 4 hours and 15-fold at 8 hours (FIG. 7C, right). P58(IPK) mRNA was induced in control but not PARP-16 knock downs (FIG. 7C, right). As expected, BiP concentrations increased at 4 hours and plateaued at 8 hours in control knock-downs due to increased transcription of BiP mRNA by spliced XBP1. A minor increase in BiP appeared at 12 hours in PARP-16 knock-downs (FIG. 7C, left).

Activation of the PERK branch appeared at 8 hours in controls as determined by PERK and eIF2α phosphorylation, and ATF4 synthesis. Such phosphorylation was barely detectable in PARP-16 knock-downs at this time-point, and PERK-dependent transcriptional programs were defective; while ATF3 and ATF4 mRNA began to accumulate at 8 hours in controls, accumulation was reduced 5-fold at 8 hours and 10-fold at 12 hours in PARP-16 knock-downs (FIG. 7C). ATF6 activation was also monitored by examining cleavage to its active transcription factor. Cleavage appeared at 4 hours in control and PARP-16 knock-downs, confirming that ATF6 activation is intact in the PARP-16 knock-downs (FIG. 7C, left).

### Example 5

### PARP-16 Directly Up-Regulates PERK and IRE1α Kinase Activity Via its (ADP-Ribosyl)Ation Activity

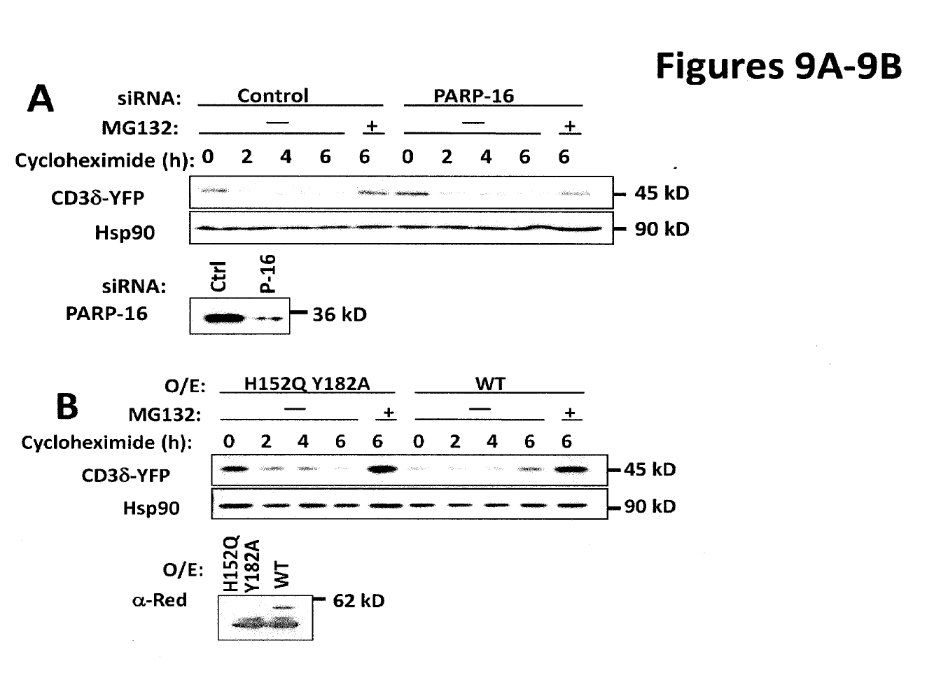

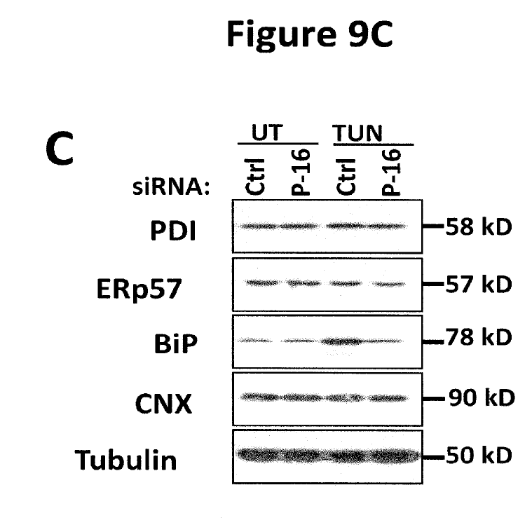

While our data strongly point to direct effects of PARP-16 on PERK and IRE1α signaling, compromised ERAD (ER-associated degradation) and/or chaperone capacities of the ER in PARP-16 knock-downs could also affect UPR activation. ERAD activity in PARP-16 knock-downs was examined by measuring clearance of CD3δ-YFP, a model substrate of ERAD machinery. CD3δ-YFP degradation kinetics were similar in PARP-16 knock-downs and controls as assayed by cycloheximide chase. Inhibition of the proteasome by MG132 rescued degradation (FIG. 9A), suggesting that ERAD activity is similar in control and PARP-16 knock-downs. Cells overexpressing intermediate amounts of mCherry-PARP-16 also exhibited similar kinetics of CD3δ-YFP clearance (FIG. 9B), suggesting that overexpression of PARP-16 does not perturb ERAD activity. The protein-folding capacity of the ER in PARP-16 knock-downs appear to be similar to controls as the protein concentrations of ER chaperones BiP and Calnexin, and disulfide isomerases PDI and ERp57, were similar (FIG. 9C).

The increase in PARP-16 enzymatic activity, and (ADP-ribosyl)ation of PERK and IRE1α during the UPR, suggests the possibility that (ADP-ribosyl)ation regulates PERK and IRE1α enzymatic activity. We examined PERK and IRE1α kinase activity in response to (ADP-ribosyl)ation by PARP-16 via self-phosphorylation assays. ER microsomes purified from GFP-PERK- or GFP-IRE1α-expressing cells were washed with 1M NaCl to remove bound PARP-16, returned to physiological salt buffer, split into duplicate reactions, and incubated with unlabeled NAD⁺ plus either GST-PARP-16 or GST-PARP-16^{H152Q Y182A}, or ³²P-NAD⁺ plus either recombinant protein. (ADP-ribosyl)ated GFP-PERK or GFP-IRE1α were extracted from the microsomes and purified under 1 M NaCl conditions to remove added recombinant PARP-16 proteins. Reactions containing unlabeled NAD⁺ were incubated with ³²P-ATP, and reactions containing ³²P-NAD⁺ were incubated with unlabeled ATP. (ADP-ribosyl)ation of GFP-PERK and GFP-IRE1α increased in a GST-PARP-16 and NAD⁺ dose-dependent manner (³²P-NAD⁺ autoradiograms in FIGS. 7D, 7E, and 8C). ³²P-NAD⁺ incorporation at the molecular weight of GST-PARP-16 was also observed (³²P-NAD⁺ autoradiograms in FIGS. 7D, 7E, and 8C), representing residual binding of GST-PARP-16 with GFP-PERK or GFP-IRE1α, even after 1 M NaCl washes. As shown in ³²P-ATP autoradiograms in FIGS. 7D, 7E, and 8C, increased (ADP-ribosyl)ation of GFP-PERK or GFP-IRE1α resulted in a dose-dependent increase in kinase activity (for GFP-PERK a 4-18 fold increase depending on NAD⁺ concentration, and for IRE1α a 2-5 fold increase depending on NAD⁺ concentration), suggesting that (ADP-ribosyl)ation by PARP-16 directly up-regulates GFP-PERK and GFP-IRE1α kinase activity. Phosphorylation by PERK at the molecular weight of GST-PARP-16 was detected in a GST-PARP-16 and NAD⁺ dose-dependent manner, indicating that PARP-16 is likely a substrate of PERK. Such phosphorylation was dramatically reduced (5-10 fold reduction depending on NAD concentration) in GST-PARP16^{H152Q Y182A} samples (FIGS. 7D and 8C). Phosphorylation by GFP-IRE1α at the molecular weight of GST-PARP-16 and GST-PARP16^{H152Q Y182A} was also detected (FIG. 7E). Such phosphorylation does not appear NAD⁺ concentration dependent.

Next, we examined the effects of (ADP-ribosyl)ation on IRE1α endonuclease activity. GFP-IRE1α purified as in FIG. 7E was incubated with ³²P-labeled mouse XBP-1 mRNA containing the intron flanked by truncated exons. Increased (ADP-ribosyl)ation of GFP-IRE1α resulted in a NAD⁺ dose-dependent cleavage of XBP-1 mRNA indicated by the appearance of 5′ and 3′ exons (5-12 fold increase, depending on NAD⁺ concentration; FIG. 7F), suggesting that (ADP-ribosyl)ation of IRE1α by PARP-16 directly up-regulates IRE1α endonuclease activity.

### Example 6

### The Luminal C-Tail of PARP-16 Senses Stress in the ER Lumen

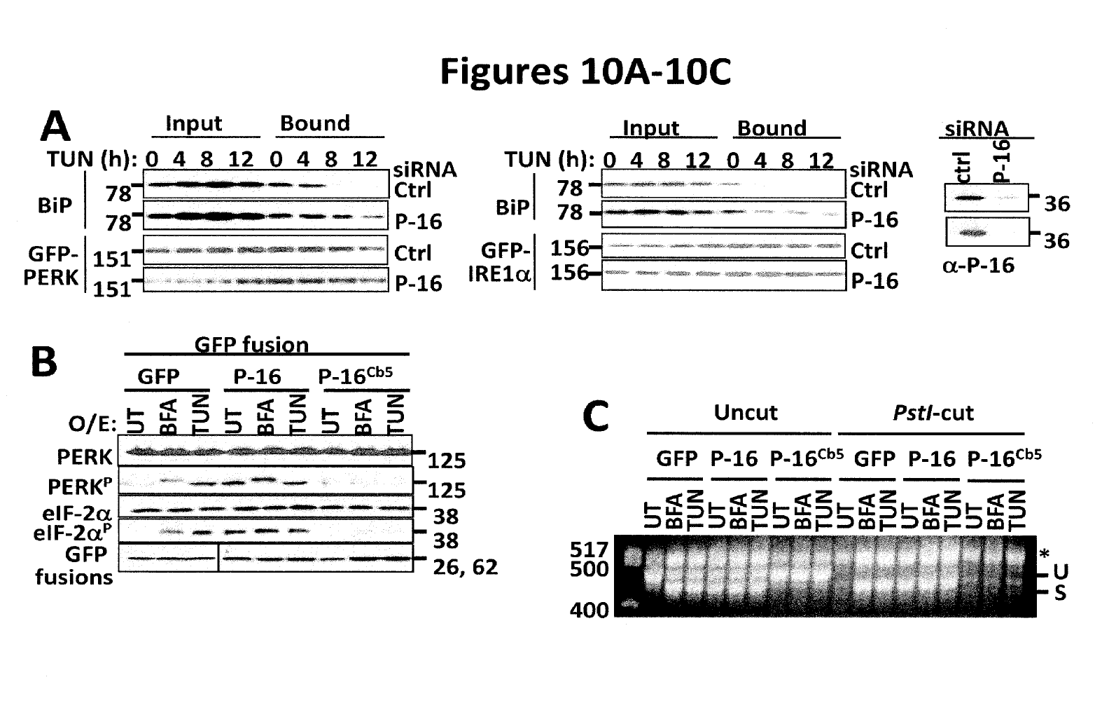

One potential mechanism by which PARP-16 regulates PERK and IRE1α is via BiP binding. We examined BiP dissociation from PERK and IRE1α to determine if it is affected in PARP-16 knock-downs. ER microsomes were purified from control or PARP-16 knock-down cells expressing either GFP-PERK or GFP-IRE1α and treated with Tunicamycin. GFP fusions were purified from the microsomes every 4 hours for 12 hours total, and immunoprecipitates analyzed for the presence of BiP. In controls, BiP dissociated from GFP-IRE1α and GFP-PERK at 4 and 8 hours, respectively (FIG. 10A). In PARP-16 knock-downs, BiP remained bound to GFP-PERK and GFP-IRE1α throughout the time course with a slight reduction in binding, suggesting that BiP dissociation was impaired (FIG. 10A). Since BiP displacement from PERK and IRE1α occurs inside the ER lumen, these data indicate a potential function for the PARP-16 C-tail in facilitating BiP dissociation from the luminal domains of PERK and IRE1α. The requirement of PARP-16 for PERK and IRE1α activation suggests that PARP-16 functions upstream of PERK and IRE1α and that the C-tail of PARP-16 might transduce stress signals from the ER lumen to the cytoplasmic PARP domain. To determine if this is the case, we expressed GFP-PARP-16^{Cb5} and treated with ER stress inducing drugs. Cells expressing GFP-PARP-16^{Cb5} were unable to activate PERK and IRE1α (FIGS. 10B, 10C, and 8B), suggesting that the luminal C-tail of PARP-16 is necessary for PARP-16 function in the UPR, and that PARP-16^{Cb5} acts as a dominant negative for PARP-16 function in the UPR.

### Example 7

### Materials and Methods

The experiments described herein may be carried out using the following materials and methods.

### Cell Culture and Transfection

HeLa and HeLa S3 cells (ATCC) were grown in DMEM (Gibco) supplemented with 10% fetal bovine serum (FBS) (Gibco) at 37° C. and 5% CO2. Cells were transfected with DNA and siRNA as described (Leung et al., Mol. Cell 42: 489-499, 2011). Stealth siRNAs (Invitrogen Inc.) directed against human PARP-16 mRNA coding region 5″-GACUUGAGCCUGGCCCUCAUAUACA-3′ (siRNA 16.3) and 5′-CCCAAGUACUUCGUGGUCACCAAUA-3′ (siRNA 16.4) were used to knock down PARP-16. siRNA 16.3 was used for all knock-down experiments, and 16.4 and 16.3 for FIG. 2D. Control siRNAs (Qiagen Allstars Negative Control siRNA) were used in parallel. All experiments involving overexpression of PARP-16 were performed at 16 hours post-transfection, except a subset of experiments described in FIG. 2C that were performed at 28 hours post-transfection. GFP-PERK, GFP-IRE1α, GFP-ATF6, and GFP-SEC6113 were expressed for 24 hours. To induce ER stress, Brefeldin A (Sigma-Aldrich), Tunicamycin (Sigma-Aldrich) and Thapsigargin (Sigma-Aldrich) were added to cell culture at 5 μg/ml, 3 μg/ml, and 0.2 μM, respectively.

### Cytological, Protein, and Immunological Techniques

Immunofluorescence analysis was performed as described in Leung et al., Mol. Cell 42: 489-499, 2011. ER-Tracker Red and a lypophilic dye DiI (Molecular Probes) were used. Trypan blue (Sigma-Aldrich) was used at 0.2%. Immunoprecipitation and immunoblotting were carried out as described in Leung et al., Mol. Cell 42: 489-499, 2011, with the exception that in some cases proteins were immunoprecipitated from ER microsomes. Fluorescence, biochemical protease protection assays and membrane extraction assays were performed as described in Schreiber et al., Nat. Rev. Mol. Cell Biol. 7: 517-528, 2006. PARP-16^{H152Q Y182A}, PARP-16^{ΔC}, and PARP-16^{AA} were generated via PCR-mediated site-directed mutagenesis using pfu polymerase (Zheng et al., Nucl. Acids Res. 32: 115, 2004). To construct the PARP-16^{Cb5} chimera, DNA sequence of the C-tail of Cytochrome b5²⁵ was added to the reverse primer for PCR. All mutations were confirmed via DNA sequencing. XBP-1 splicing assays were performed as described²⁶. Antibodies used: PARP-16 (Aviva ARP33751; Cocalico Biologicals, custom made, HM933), Calnexin (BD, 610523), Lamin B1 (Abeam, ab20396), Lamin B2 (Abeam, ab8983), Lamin A/C (Abeam, ab8984), Tubulin (Abeam, ab6161), GFP (Invitrogen, A11120; Rockland, 600-401-215), Red (Chromotek, 5F8), PERK (Sigma-Aldrich, HPA015737, P0074; Santa Cruz, sc-9481), phospho-PERK (Santa Cruz, sc-32577), eIF2α (Abeam, ab50733), phospho-eIF2α (Sigma-Aldrich, SAB4300221), IRE1α (Cell Signaling, 3294), phospho-IRE1α (Thermo Scientific, PA1-16927), ATF4 (Abeam, ab1371), XBP-1 (Abeam, ab37152), PDI (Abeam, ab2792), ERp57 (Abeam, ab 10287), BiP (Cell Signaling, 3177), ATF6 (Abeam, ab11909), Mannosidase II (Abeam, ab12277), Hsp90 (Stressgen, SPA-830), p230 (gift from F. Gertler), EEA1 (Abeam, ab15846), MTCO2 (Abeam, ab3298), phospho-γH2AX (Millipore, 05-636), TIA-1 (Santa Cruz, sc-1751). Antibodies were used at 1:1,000 for immunoblotting (except for anti-eIF2α used at 1:50); 1:500 for immunoprecipitation; and 1:100 for immunofluorescence.

### NAD⁺ Incorporation Assays

For ER microsome-based assays including NAD⁺ incorporation and immunoprecipitation, ER microsomes were fractionated from HeLa cells via isopycnic flotation method as described in Stephens et al., Methods Mol Biol. 419: 197-214, 2008, and incubated with 100 μM β-NAD⁺ (MP Biomedicals) and 2.5 μCi of ³²P-NAD⁺ (Perkin Elmer) in a PARP reaction buffer (50 mM HEPES pH 7.4, 150 mM NaCl, 1 mM MgCl₂, 10 mM EGTA, 1 mM DTT, 0.1 mM Na₃VO₄, 50 mM NaF, 5 mM β-glycerophosphate, 1 μM ADP-HPD, and protease inhibitor cocktail) at 25° C. for 30 min. The ratio between unlabeled and labeled NAD⁺ was kept constant upon titration of NAD⁺ concentration. The ³²P-labeled ER microsomes were then lysed by addition of Triton X-100 at 1%. Proteins were immunoprecipitated, eluted in a 1× Laemmli sample buffer by boiling at 65° C. for 10 min, and then analyzed using autoradiogram and immunoblotting. For assays using recombinant proteins, GST-PARP-16 wild-type and mutant isoforms were purified from BL 21 RIPL cells, following manufacturer's protocol (Stratagene). NAD⁺ incorporation reactions were performed under the same conditions described above.

### Kinase Assays

PERK and IRE1α kinase assays were performed as described herein. Notably, the kinase activities were measured post-NAD⁺ incorporation by PARP-16 in the context of ER microsomes. In brief, ER microsomes were subject to NAD incorporation assays in a PARP buffer as described in the NAD⁺ Incorporation Assays section using 100 μM unlabeled NAD⁺ (MP Biomedicals) and GST-PARP-16 wild-type or catalytically inactive mutant proteins purified from bacteria. The (ADP-ribosyl)ated ER microsomes were incubated with 100 μM ATP (New England BioLabs) and 2.5 μCi of [γ-³²P]ATP (Perkin Elmer) in a kinase buffer (for GFP-PERK activity, 20 mM HEPES pH 7.4, 50 mM KCl, 1.5 mM DTT, 2 mM Mg(OAc)₂, 2 mM MnCl₂, 0.1 mM Na₃VO₄, 50 mM NaF, 5 mM β-glycerophosphate, and protease inhibitor cocktail; for GFP-IRE1α activity, 20 mM HEPES pH 7.4, 1 mM DTT, 10 mM Mg(OAc)₂, 50 mM K(OAc)₂, 0.1 mM Na₃VO₄, 50 mM NaF, 5 mM β-glycerophosphate, and protease inhibitor cocktail) at 25° C. for 30 min. The ³²P-labeled ER microsomes were then lysed by addition of Triton X-100 at 1%. Proteins were immunoprecipitated, eluted in 1× Laemmli sample buffer by heating at 65° C. for 10 min, and then analyzed using autoradiogram and immunoblotting.

### IRE1α Endonuclease Assays

A 479-bp mouse XBP-1 DNA fragment containing the intron and flanking exons on both sides (263 bp on the 5′ end and 191 bp on the 3′ end) was amplified via PCR using a reverse primer containing T7 RNA polymerase promoter sequence. In vitro transcription of XBP-1 mRNA and XBP-1 cleavage assays were performed and gel-purified transcript equivalent to ˜20,000 cpm was incubated with (ADP-ribosyl)ated GFP-IRE1α immunoprecipitates in an IRE 1α kinase buffer plus 2 mM unlabeled ATP at 25° C. for 30 min. The cleavage products were analyzed on 10% TBE-UREA polyacrylamide gels.

### ROS Generation and Ca²⁺ Measurement

ROS was evaluated using a cell permeant carboxymethyl derivative of fluorescein (CM-H₂DCFDA, Invitrogen), following the manufacturer's protocol (Invitrogen). Cells were loaded with CM-H₂DCFDA at 5 μM for 30 min at 37° C., then treated with H₂O₂ at 100 μM for 10 min at 37° C. The fluorescence intensity of oxidized ROS probe was measured using a microplate reader (Tecan). To measure intracellular Ca²⁺ concentration, cells were loaded with a cell permeant Ca²⁺ probe, Fura-4F (Invitrogen) at 2 μM in the Krebs-Ringer solution containing HEPES and 2 mM CaCl₂ for 30 min at 37° C. The ratiometric fluorescence at 340 nm/380 nm was measured every 2 min for 20 min, using a microplate reader (Tecan). Thapsigargin was added at 1 μM, and EGTA used at 3 mM to chelate the released Ca²⁺. To induce cytoplasmic stress and DNA damage, Arsenite (Sigma-Aldrich) and Cisplatin (Sigma-Aldrich) were used at 100 μM and 10 μM, for 30 min and 8 h, respectively. To inhibit translation and the proteasome, Cycloheximide (Sigma-Aldrich) and MG132 (Sigma-Aldrich) were used at 100 μg/ml and 10 μM, respectively. Total RNA was extracted using RNeasy kits and QIAshredder (Qiagen), and cDNA was amplified using Quantitect reverse transcription kit (Qiagen). RT-qPCR was performed with Quantitect SYBR Green PCR kit (Qiagen), using a LightCycler 480 (Roche). mRNA levels were normalized against GAPDH mRNA.

### Statistics

All experiments were repeated a minimum of two times and the unpaired Student's t-test was used for statistical analysis.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

All patents, patent applications, patent application publications, and other publications cited or referred to in this specification are hereby incorporated by reference to the same extent as if each independent patent, patent application, patent application publication, or publication was specifically and individually indicated to be incorporated by reference. Such patent applications specifically include U.S. Provisional Patent Application No. 61/552,210, filed Oct. 27, 2011, and U.S. Provisional Patent Application No. 61/715,758, filed Oct. 18, 2012, from which this application claims benefit.

1-32. (canceled)

33. A method of treating a subject with an ER stress-related condition, the method comprising administering to the subject a therapeutically effective amount of a pharmaceutical composition that decreases PARP-16 expression or activity.

34. The method of claim 33, wherein the ER stress-related condition is a cancer, protein folding/misfolding disease, diabetes mellitus, Wolcott-Rallison syndrome, ischemia/reperfusion injury, stroke, neurodegeneration, atherosclerosis, neoplasia, hypoxia, or hypoglycemia.

35. The method of claim 33, wherein the pharmaceutical composition comprises a PARP-16-specific inhibitor.

36. The method of claim 35, wherein said inhibitor is an RNA aptamer, a small molecule, or an antibody.

37. The method of claim 36, wherein said antibody binds to a cytoplasmic domain of PARP-16.

38. The method of claim 37, wherein said antibody binds at or near an active site within the cytoplasmic domain of PARP-16.

39. The method of claim 36, wherein said antibody has a Kd equal to or less than 10 μM.

40. The method of claim 36, wherein said small molecule inhibits PARP-16 activity by reducing NAD⁺ substrate occupancy of a PARP-16 active site.

41. A method of treating a subject with an ER stress-related condition, the method comprising administering to the subject a therapeutically effective amount of a pharmaceutical composition that increases PARP-16 expression or activity.

42. The method of claim 41, wherein the ER stress-related condition is a myelinating cell-related disease, protein folding/misfolding disease, or bipolar disorder.

43. The method of claim 41, wherein the pharmaceutical composition comprises a PARP-16-specific activator.

44. The method of claim 33 or 41, wherein the pharmaceutical composition further comprises a pharmaceutically acceptable carrier.

45. The method of claim 33 or 41, wherein the pharmaceutical composition is administered intramuscularly, intravenously, intradermally, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctival, intravesicularlly, mucosally, intrapericardially, intraumbilically, intraocularally, orally, topically, locally, by inhalation, injection, infusion, continuous infusion, localized perfusion bathing target cells directly, catheter, lavage, in cremes, or lipid compositions.

46. A method of diagnosing an endoplasmic reticulum (ER) stress-related condition in a subject, the method comprising analyzing the level of poly(ADP-ribose) polymerase 16 (PARP-16) expression or activity in a sample isolated from the subject, wherein an increased level of PARP-16 expression or activity in the sample relative to the level in a control sample indicates that the subject has the ER stress-related condition.

47. A method of identifying a candidate compound useful for treating a subject having an ER stress-related condition, the method comprising:
(a) contacting a PARP-16 protein, or fragment thereof, with a compound; and
(b) measuring the activity of the PARP-16, wherein a decrease in PARP-16 activity in the presence of the compound identifies the compound as a candidate compound for treating an ER stress-related condition in a subject.

48. A method of identifying a candidate compound useful for treating a subject having an ER stress-related condition, the method comprising:
(a) contacting a PARP-16 protein, or fragment thereof, with a compound; and
(b) measuring the activity of the PARP-16, wherein an increase in PARP-16 activity in the presence of the compound identifies the compound as a candidate compound for treating an ER stress-related condition in a subject.

49. A kit for treating a subject with an ER stress-related condition, the kit comprising:
(a) a pharmaceutical composition that modulates PARP-16 expression or activity; and
(b) instructions for administering the pharmaceutical composition to the subject.


SEQUENCE LISTING (2 sequences)

SEQ ID NO: 1 - RNA, 25 nt, Artificial Sequence
  Synthetic Construct
gacuugagccuggcccucauauaca

SEQ ID NO: 2 - RNA, 25 nt, Artificial Sequence
  Synthetic Construct
cccaaguacuucguggucaccaaua